(19) United States
(12) Patent Application Publication
(10) Pub. No.: US 2025/0205230 A1
(43) Pub. Date: Jun. 26, 2025

(54) TREATMENT OF VIRAL INFECTIONS

(71) Applicants: Ramot at Tel-Aviv University Ltd., Tel-Aviv (IL); The Regents of The University of California, Oakland, CA (US)

(72) Inventors: Gerardo Zelmar LEDERKREMER, Tel-Aviv (IL); Marcelo EHRLICH, Tel-Aviv (IL); Marina SHENKMAN, Tel-Aviv (IL); Elvira HAIMOV, Tel-Aviv (IL); Raul ANDINO-PAVLOVSKY, San Francisco, CA (US); Yinghong XIAO, San Francisco, CA (US); Ranen AVINER, San Francisco, CA (US); Petr V. LIDSKIY, San Francisco, CA (US)

(73) Assignees: Ramot at Tel-Aviv University Ltd., Tel-Aviv (IL); The Regents of The University of California, Oakland, CA (US)

(21) Appl. No.: 18/852,465

(22) PCT Filed: Mar. 29, 2023
(86) PCT No.: PCT/IL2023/050341

§ 371 Date: Sep. 29, 2024

Related U.S. Application Data
(60) Provisional application No. 63/324,975, filed on Mar. 29, 2022.

(51) Int. Cl. A61K 31/505

(52) U.S. Cl. CPC A61K 31/505

(57) ABSTRACT

Compounds capable of activating an unfolded protein response (UPR) in a cell, for use in treating a viral infection in a subject in need thereof, are disclosed. The compounds can be PERK activators and/or compounds represented by Formula I as described in the specification.

### RELATED APPLICATION/S

This application claims the benefit of priority of U.S. Patent Application No. 63/324,975 filed on Mar. 29, 2022, the contents of which are incorporated herein by reference in their entirety.

### FIELD AND BACKGROUND OF THE INVENTION

The present invention, in some embodiments thereof, relates to therapy, and more particularly, but not exclusively, to compounds usable in the treatment of viral infections.

Many viruses, coronaviruses included, are dependent on cellular resources—such as endoplasmic reticulum (ER) function and membranes—for the synthesis of viral proteins, assembly, egress and manipulation of antiviral responses [Oostra et al. (2007) J Virol, 81(22), 12323-12336; Stertz et al. (2007) Virology, 361(2), 304-315; Reggiori et al. (2010) Cell Host Microbe, 7(6), 500-508; Knoops et al. (2008) PLoS Biol, 6(9), e226]. For instance, the coronavirus SARS-CoV-2 encodes multiple membrane glycoproteins, generates unique double membrane vesicles (DMVs) as viral replication compartment, and employs the ER-Golgi intermediate compartment as budding site. In this context, coronaviruses and many other viruses induce the unfolded protein response (UPR), an ER stress response to glycoprotein overload [Minakshi et al. (2009) PLoS One, 4(12), e8342; Fung et al. (2019) Virology, 533, 34-44; Fung et al. (2014) Front Microbiol, 5, 296; Fung et al. (2014) Virus Res, 194, 110-123; Chan et al. (2006) J Virol, 80(18), 9279-9287].

This feedback loop is termed integrated stress response (ISR). It can be elicited by four different eIF2α kinases (PERK, PKR, GCN2 and HRI), each can be activated by different stressors. PKR is activated by double stranded RNA [Gal-Ben-Ari et al. (2018) Front Mol Neurosci, 11, 480], an obligatory byproduct of RNA virus replication, which provides an overlap between antiviral and stress-response cellular programs.

The ISR process starts with phosphorylation of translation initiation factor eIF2α to inhibit its guanine exchange factor eIF2B, thereby causing global reduction of protein synthesis; a mechanism that is circumvented by many viruses [Jan et al. (2016) Annu Rev Virol, 3(1), 283-307]. While UPR-derived chaperone expression upregulation and metabolism/redox changes are expected to benefit viral infection, translational attenuation is predicted to be deleterious.

This regulatory crossroad was found to be central in coronavirus infection. For example, TGEV (an alpha-coronavirus) replication is suppressed via concomitant PERK-eIF2α-mediated inhibition of viral proteins synthesis, and NF-κB-induced IFN-I production. The ISR inhibitor ISRIB increased expression of TGEV viral proteins and viral titers [Xue et al. (2018) J Virol, 92(15)].

A recent SARS-CoV-2 proteomics study identified cellular targets bound by viral proteins [Gordon et al. (2020) bioRxiv. 2020.03.22.002386].

The metabolic and regulatory challenges posed by high expression of viral membrane proteins and the stress response it elicits, are predicted to be a common factor in the replication of all enveloped viruses that reach high titers in cells (e.g., RNA viruses). Moreover, the elicitation of innate immune antiviral responses (in which PKR activation plays a central role) is also a common feature of the cellular response to multiple different viruses. As such, the discovery of compounds which affect cellular mechanisms involved in stress- or antiviral-responses hold the potential of functioning as “broad-range” antivirals.

Small molecules modulators of PERK, such as GSK2606414, GSK2656157 and A4 (structure shown below), have been designed primarily as cancer-treating drugs, but also as candidates for treating neurodegenerative diseases [see for example, Wang et al. (2010) supra; Axten et al. (2012) J Med Chem, 55: 7193-7207; Axten et al. (2013) ACS Med Chem Lett, 4:964-968; Moreno et al. (2013) Sci Transl Med, 5(206):206ra138; Radford et al. (2015) Acta Neuropathol, 130:633-642; and International Patent Application Publications WO 2011/119663 and WO 2011/146748].

Additional hydrazone derivatives such as MK-28 have been found to modulate protein kinase R (PKR)-like endoplasmic reticulum kinase (PERK) [see, for example, Ganz et al. (2020) Scientific Reports, 10, 6875; and U.S. Pat. No. 11,104,651], and as usable in treating Huntington's disease, as well as other conditions associated with protein aggregation [see, for example, WO 2017/216792 and Ganz et al. (2020) supra].

Additional background art includes U.S. Pat. No. 11,104,651; Dellac et al. (2021) Int J Cancer 148, 2321-2334; Wang et al. (2010), Chem Biol Drug Des, 76, 480-495; Bruch et al. (2016) EMBO Mol Med, 9, 371-384.

### SUMMARY OF THE INVENTION

According to an aspect of some embodiments of the invention, there is provided a compound for use in treating a viral infection, the compound being capable of activating an unfolded protein response in a cell, thereby treating the viral infection.

According to some of any of the embodiments described herein, the compound is a PERK activator.

According to an aspect of some embodiments of the invention, there is provided a compound for use in treating a viral infection, the compound being a PERK activator.

According to some of any of the embodiments described herein, the compound is represented by Formula I:

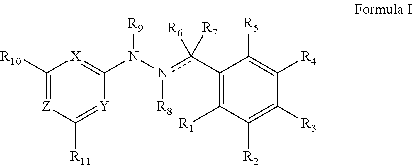

or a pharmaceutical acceptable salt thereof,
wherein:
the dashed line denotes a saturated or unsaturated bond;
X is N or CR₁₂;
Y is N or CR₁₃;
Z is N or CR₁₄;
R₁-R₇ and R₁₀-R₁₄ are each independently selected from the group consisting of hydrogen, alkyl, alkenyl, alkynyl, cycloalkyl, aryl, heteroaryl, heteroalicyclic, halo, hydroxy, alkoxy, aryloxy, thiohydroxy, thioalkoxy, thioaryloxy, sulfinyl, sulfonyl, sulfonate, sulfate, cyano, nitro, azide, phosphonyl, phosphinyl, carbonyl, thiocarbonyl, a urea group, a thiourea group, O-carbamyl, N-carbamyl, O-thiocarbamyl, N-thiocarbamyl, S-thiocarbamyl, C-amido, N-amido, C-carboxy, O-carboxy, sulfonamido, guanyl, guanidinyl, hydrazine, hydrazide, thiohydrazide, and amino; and
R₈ and R₉ are each independently hydrogen or alkyl,
wherein when the dashed line denotes an unsaturated bond, R₇ and R₈ are absent.

According to some of any of the embodiments described herein, R₁ is hydrogen or hydroxy.

According to some of any of the embodiments described herein, R₁ is hydrogen.

According to some of any of the embodiments described herein, R₂-R₄ are each independently selected from the group consisting of hydrogen, hydroxy, alkoxy, and alkyl.

According to some of any of the embodiments described herein, at least one of R₂-R₄ is hydroxy.

According to some of any of the embodiments described herein, R₂ and R₃ are each hydroxy.

According to some of any of the embodiments described herein, at least one of R₂-R₄ is alkoxy.

According to some of any of the embodiments described herein, at least one of R₂-R₄ is trifluoromethyl.

According to some of any of the embodiments described herein, R₅ and R₆ are each hydrogen.

According to some of any of the embodiments described herein, the dashed line denotes a saturated bond and R₇ and R₈ are each hydrogen, or the dashed line denotes an unsaturated bond and R₇ and R₈ are each absent.

According to some of any of the embodiments described herein, the dashed line denotes an unsaturated bond.

According to some of any of the embodiments described herein, R₁₀ and R₁₁ are each independently selected from the group consisting of hydrogen, alkyl, cycloalkyl, aryl, heteroaryl, and heteroalicyclic.

According to some of any of the embodiments described herein, R₁₀ and R₁₁ are each independently a substituted or non-substituted phenyl.

According to some of any of the embodiments described herein, R₉ is hydrogen or methyl.

According to some of any of the embodiments described herein, R₁₂-R₁₄ are each hydrogen.

According to some of any of the embodiments described herein, at least one of X, Y and Z is nitrogen.

According to some of any of the embodiments described herein, X and Y are each nitrogen.

According to some of any of the embodiments described herein, Z is CH.

According to some of any of the embodiments described herein, the viral infection is associated with a virus selected from the group consisting of double strand DNA viruses, single strand DNA viruses, double strand RNA viruses, (+)-single strand RNA viruses, (−)-single strand RNA viruses, RNA retroviruses; DNA retroviruses, satellite viruses, and viroids.

According to some of any of the embodiments described herein, the virus is an RNA virus.

According to some of any of the embodiments described herein, the virus is selected from the group consisting of coronaviruses, rhabdoviruses and reoviruses.

According to some of any of the embodiments described herein, the virus is a Severe acute respiratory syndrome coronavirus (SARS-CoV) or a Vesicular stomatitis Indiana virus (VSV).

According to some of any of the embodiments described herein, the treating results in inhibition of exit of a viral glycoprotein from endoplasmic reticulum.

According to some of any of the embodiments described herein, the compound forms a part of a pharmaceutical composition which further comprises a pharmaceutically acceptable carrier.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

### BRIEF DESCRIPTION OF THE SEVERAL VIEWS OF THE DRAWING(S)

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIG. 1 presents comparative bar graphs showing the influence of treatment with MK-28 (10 μM), an exemplary PERK activator according to some of the present embodiments, on the titer of infectious virions in Vero E6 cells infected with SARS-CoV-2, with 0.05, 0.5 or 5 multiplicity of infections (MOI), for 1 hour at 4° C. Cells were incubated for 3 hours with MK-28 pre-infection, and were post-infection re-treated in a medium containing MK-28 for a period of either 16 or 24 hours post-infection (hpi) at 37° C. Treatment with DMSO served as a control.

FIG. 2 presents bar graphs depicting viral titers of the supernatants of HEK293 cells, untreated (UT) or treated with the exemplary compounds MK-28 or GLB-7 (10 μM), 24 hours after infection with VSVΔ51M (with 0.1 or 1 MOI).

FIGS. 3A-B present an image of a western blot of VSV-G protein of VSVΔ51M-infected HEK293 cells (0.1 or 1 MOI), with or without pre-incubation with the exemplary compound MK-28 (10 μM), the cells being lysed 24 hours post-infection with or without treatment of the cell lysate with Endo H (FIG. 3A), and a bar graph showing the percentage of Endo H-resistant VSV-G protein (FIG. 3B).

FIGS. 4A-4C present images of representative Western blots of HEK293 cells transfected with plasmids expressing VSV-G (FIG. 4A), asialoglycoprotein receptor H2b (FIG. 4B) and ACE2 (FIG. 4C), and lysed 24 hours post-transfection, the lysates being untreated (UT) or treated with Endo H (FIGS. 4A-4C) or N-glycanase (FIG. 4C).

FIGS. 5A-E present images of representative Western blots (FIG. 5A) and respective quantifications (FIG. 5B-E), showing the influence of treatment with MK-28 (10 μM or 20 μM, as indicated), an exemplary PERK activator according to some of the present embodiments, on the levels of eIF2α-phosphorylation (eIF2α-P) relatively to total eIF2α, and VSV-G relatively to actin levels in HEK293 cells infected at MOI of 0.1 with VSVΔ51M, lysed 24 hours post-infection. The treatment with MK-28 began either concomitantly with the infection (marked “same time as infection”; FIGS. 5B-C) or 3 hours (3 h) after the start of the infection (FIGS. 5D-E), as indicated. Immunoblotting was performed with anti-eIF2α-P, anti-total eIF2α, anti-VSV-G or anti-actin antibodies.

FIG. 6 presents bar graphs showing the influence of treatment with MK-28 (10 μM or 20 μM, as indicated), an exemplary PERK activator according to some of the present embodiments, on the levels of eIF2α-phosphorylation (eIF2α-P) relatively to total eIF2α (eIF2α) in lysed HEK293 cells. Treatment with MK-28 began 24 hours before lysis. Data was obtained from quantification of Western blots (not presented), and immunoblotting was performed with anti-eIF2α-P or anti-total eIF2α antibodies.

FIGS. 7A-C present images of representative Western blots (FIG. 7B) and respective quantifications (FIGS. 7A and 7C), showing the influence of treatment with GLB7 (10 μM or 20 μM, as indicated), an exemplary PERK activator according to some of the present embodiments, on the levels of eIF2α-phosphorylation (eIF2α-P) relatively to total eIF2α (FIG. 7A) and VSV-G level relatively to actin level (FIG. 7C) in HEK293 cells infected at MOI of 0.1 with VSVΔ51M and lysed 24 hours post-infection. Treatment with GLB7 began concomitantly with the infection. The data in FIG. 7C was obtained from quantification of Western blots (see, FIG. 7B; Western blots of eIF2α-P and total eIF2α are not presented), and immunoblotting was performed with anti-eIF2α-P, anti-total eIF2α, anti-VSV-G or anti-actin antibodies.

### DESCRIPTION OF SPECIFIC EMBODIMENTS OF THE INVENTION

The present invention, in some embodiments thereof, relates to therapy, and more particularly, but not exclusively, to compounds usable in the treatment of viral infections.

Before explaining at least one embodiment of the invention in detail, it is to be understood that the invention is not necessarily limited in its application to the details set forth in the following description or exemplified by the Examples. The invention is capable of other embodiments or of being practiced or carried out in various ways.

Many viruses, including coronaviruses such as SARS-CoV-2, modulate the cellular integrated stress response (ISR), for the benefit of viral infectivity and proliferation. Activation of PKR (protein kinase R), phosphorylates the translation factor eIF2α, leading to a global inhibition of protein synthesis, which affects synthesis of viral proteins. Many viruses attack this process by inhibiting PKR.

A novel therapeutic strategy is described herein, whereby viral infectivity is blocked by targeting a central component of the ISR, the ER membrane unfolded protein response (UPR) sensor PERK. Activation of PERK, which, similarly to the cytosolic PKR, causes eIF2α phosphorylation, can boost the ISR and reduce viral protein synthesis.

While reducing the present invention to practice, exemplary small molecule PERK activators were shown to exhibit activity and selectivity in cells and in vivo in mice, with no apparent toxicity. Infectivity assays performed in cells suggest efficient cell protection against VSV and SARS-CoV-2 viruses. A reduction in VSV protein maturation was observed, which may serve as a mechanism for hampering infectivity, via reduced viral budding and/or inclusion of immature forms of the G protein into virions. These results indicate that the novel therapeutic strategy described herein is applicable to a broad range of viruses.

Without being bound by any particular theory, it is believed that many viral infections (e.g., SARS-CoV-2 infection) require a delicate balance between (i) enhanced viral protein synthesis, folding and processing at the ER of infected cells; and (ii) the stress response that follows such increased synthesis. This balance facilitates viral manipulation of cellular defenses, replication and generation of infectious particles. It is further believed that PERK activators can exhibit an antiviral effect by disrupting such a balance in a manner that favors cellular antiviral defenses over replication ability; the ISR may be boosted at the expense of viral proliferation, and thus reduce infectivity.

As this approach does not rely on a particular viral target involved in binding or infection, it is believed that it will be applicable to the treatment of a broad range of viral infections and not be limited to specific viral variants.

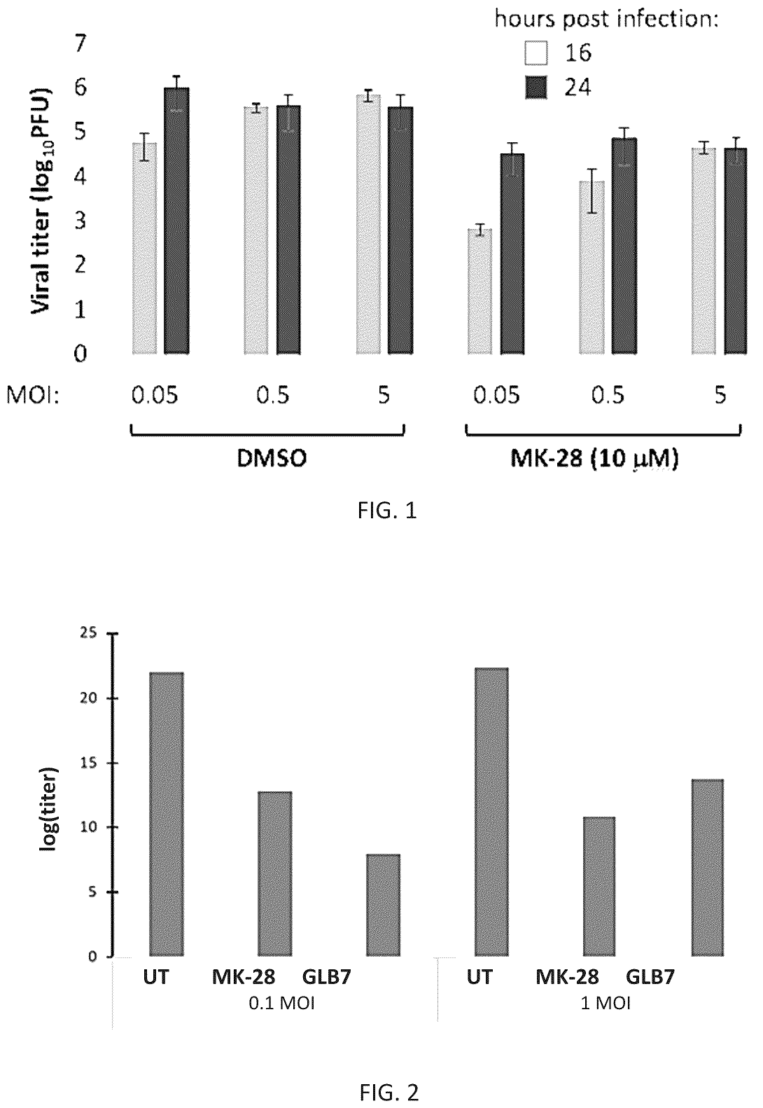

The present inventors have studied the effect of the exemplary compounds MK-28 and GLB7 on the replication of SARS-CoV-2 and VSVΔ51M (vesicular stomatitis Indiana virus; VSV) in infected Vero E6 or HEK293 cells, respectively. FIGS. 1 and 2 present the data obtained in these studies. The present inventors have uncovered that activation of PERK (using, e.g., the exemplary compounds) can result in antiviral activity against a variety of viruses.

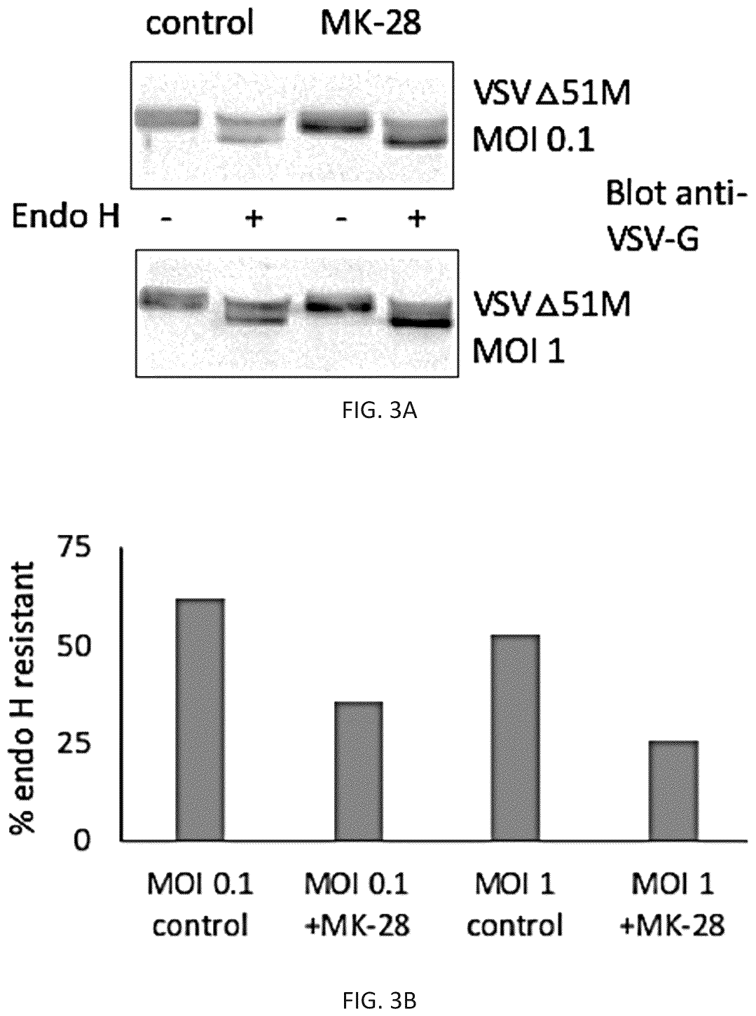

The present inventors have studied the mechanism of action of the observed inhibition of replication by the exemplary compound MK-28. FIGS. 3A-B present the data obtained in these studies. The present inventors have uncovered that PERK activation (using, e.g., the exemplary compounds) promotes ER retention in cells infected by viruses, which inhibits viral maturation.

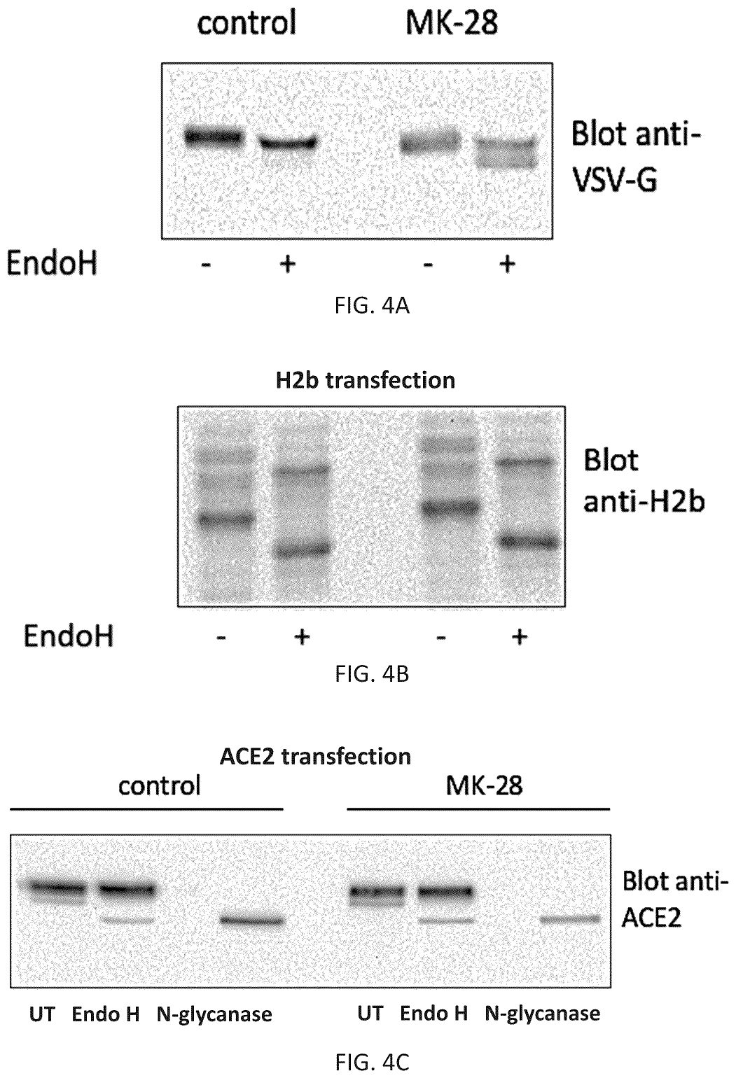

The present inventors have studied the specificity of the exemplary compounds in inhibiting maturation of viral proteins. The data obtained in this study are presented in FIGS. 4A-C, and have uncovered that inhibition of glycoprotein maturation by PERK activators is specific to viral proteins. It has been assumed that the antiviral effect of the PERK activators stems from effects on virus replication, and not on trafficking defects of, e.g., the ACE2 receptor. Without being bound by any particular theory, it is believed that the ability of the PERK activators to suppress virus replication stems from their ability to activate an unfolded protein response in a cell.

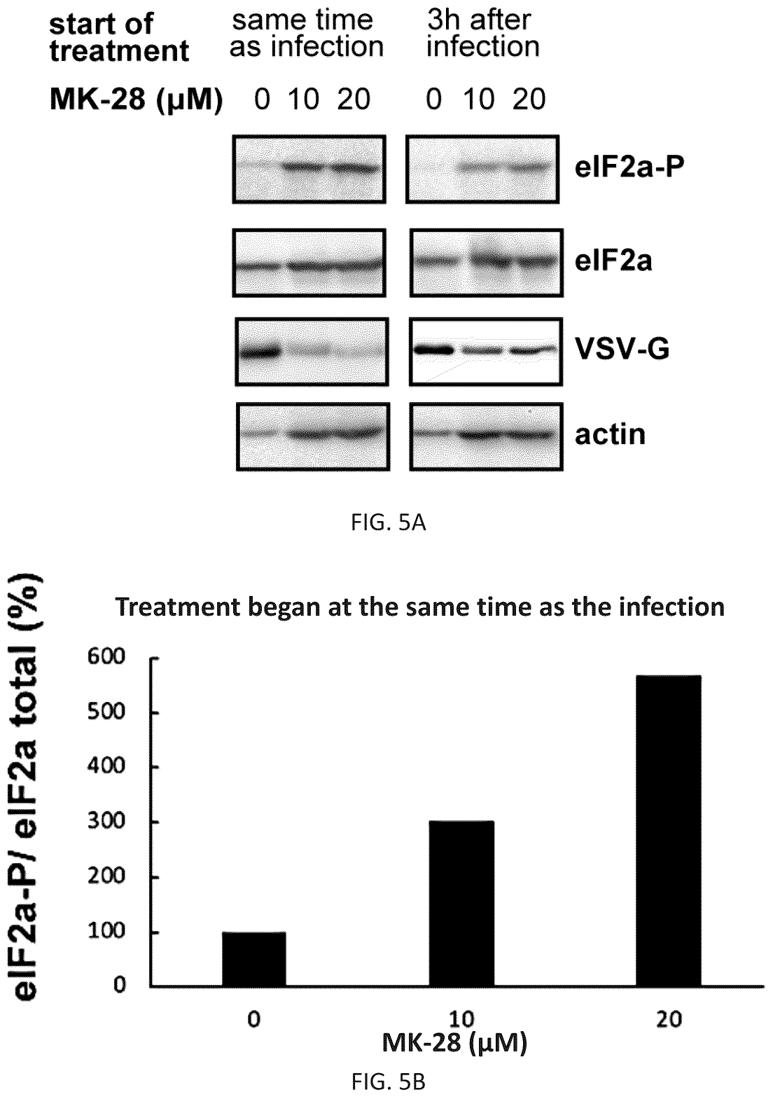

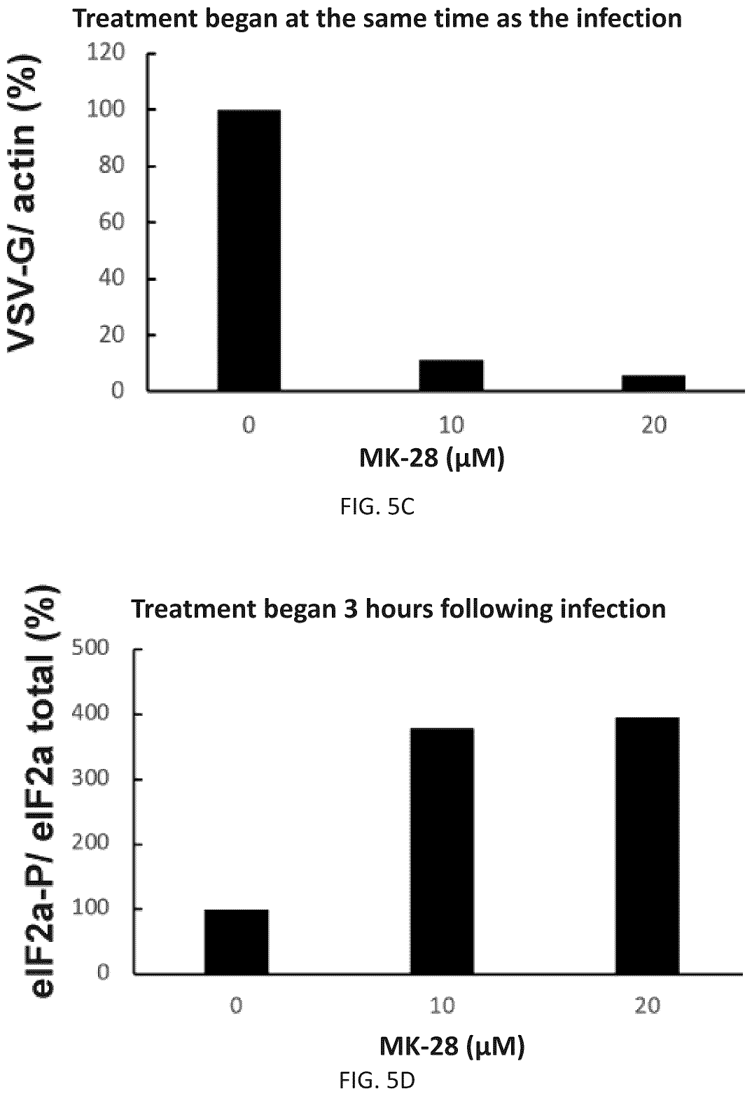

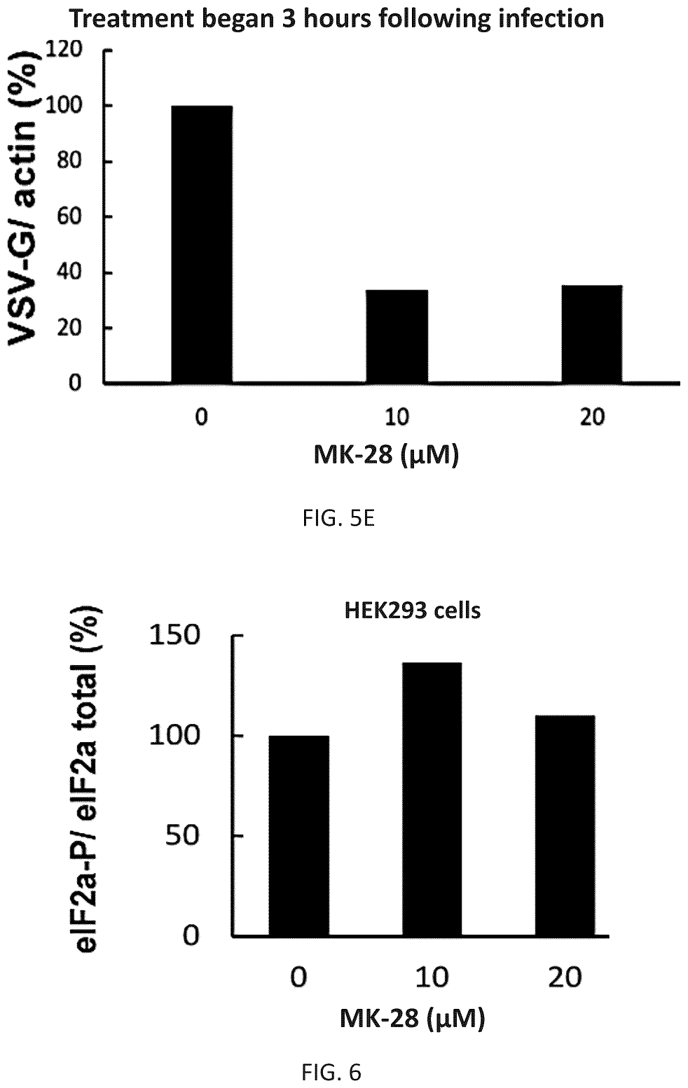

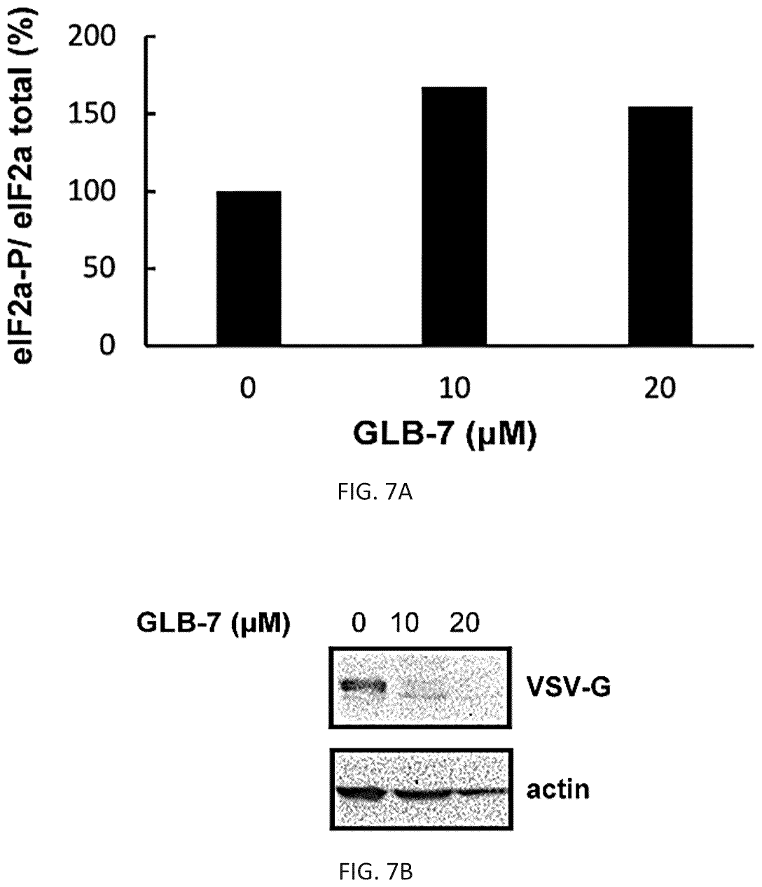

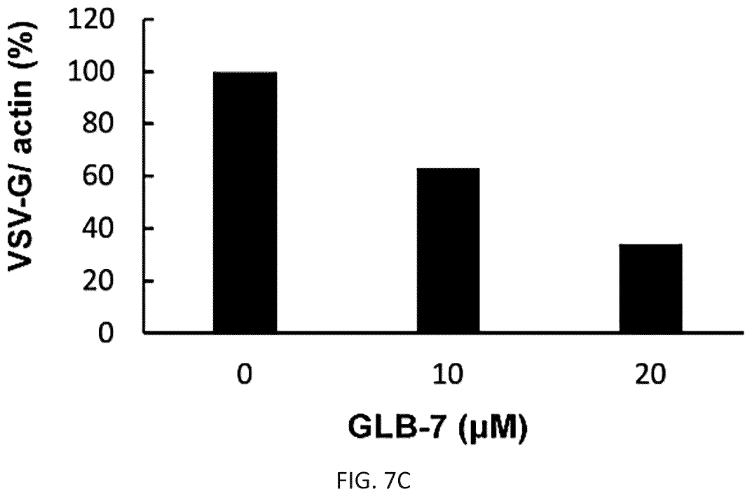

The present inventors have then studied in-vitro the activity of PERK under viral infection following treatment with exemplary PERK activators, and have uncovered that the exemplary compounds both activate PERK and decrease infectivity in viral-infected cells. The obtained data are presented in FIGS. 5A-E and FIGS. 7A-C. It has been also demonstrated that exemplary PERK activators exhibit higher activation of PERK in viral-infected cells compared to non-infected cells (FIG. 6).

Embodiments of the present invention relate to the use of compounds that are capable of activating an unfolded protein response in a cell, and/or which are PERK activators, in treating a viral infection. In some of any of the embodiments described herein, the compounds are represented by Formula I, as described herein in any of the respective embodiments and any combination thereof. According to an aspect of some embodiments of the present invention there is provided a compound for use in treating a viral infection in a subject in need thereof. According to some embodiments, the compound is such that is capable of modulating (e.g., activating) an unfolded protein response (UPR), e.g., in a cell, thereby treating the viral infection.

In some of any of the embodiments described herein, the compound is such that is capable of modulating (e.g., activating) an unfolded protein response (UPR) in an infected cell, e.g., a cell infected by viral infection, and/or in a viral cell.

Herein, the terms “unfolded protein response” and “UPR” refer to a cellular stress response associated with endoplasmic reticulum (ER) stress, which includes at least three components that counteract ER stress: stress gene expression, translational attenuation, and ER-associated protein degradation (ERAD). UPR typically includes activity by the transducer proteins XBP1, ATF6 and PERK.

Herein, “modulating an unfolded protein response” means affecting a degree of any (optionally all) of the processes and/or protein activities encompassed by an unfolded protein response, for example, by affecting an amount of a protein involved in the unfolded protein response and/or by modulating the protein's activity. Modulating may optionally be effected on the genomic and/or the transcript level by affecting transcription and/or translation of one or more proteins involved in an unfolded protein response; and/or on the protein's level by modulating an activity of one or more proteins involved in an unfolded protein response (e.g., by phosphorylation/dephosphorylation, agonism, antagonism, stabilizing or destabilizing the protein, and the like).

Preferably, the compound is such that is capable of activating or upregulating an unfolded protein response (UPR) in an infected cell.

Herein, “activating an unfolded protein response” and “upregulating an unfolded protein response”, which are used interchangeably, mean increasing a degree of any (optionally all) of the processes and/or protein activities encompassed by an unfolded protein response, for example, by increasing an amount of a protein involved in the unfolded protein response and/or by activating the protein. Upregulating may optionally be effected on the genomic and/or the transcript level by promoting transcription and/or translation of one or more proteins involved in an unfolded protein response; and/or on the protein's level by activating one or more proteins involved in an unfolded protein response (e.g., by phosphorylation/dephosphorylation, agonism, preventing cleavage of the protein, and the like).

Modulation of UPR may optionally be determined as an increase or a decrease in phosphorylation of eIF2α, for example, in the presence of a condition or compound (e.g., a compound as described herein in any of the respective embodiments) which induces integrated stress response (ISR) by, e.g., phosphorylation of translation initiation factor eIF2α (e.g., as exemplified herein). Activation or upregulation of UPR may optionally be determined as an increase in phosphorylation of eIF2α, for example, in the presence of a condition or compound (e.g., a compound as described herein in any of the respective embodiments) which induces integrated stress response (ISR) by, e.g., phosphorylation of translation initiation factor eIF2α (e.g., as exemplified herein).

According to some of any of the embodiments described herein, the compound is a modulator of (e.g., is capable of modulating) any one of XBP1, ATF6 and PERK. In some of any of the embodiments described herein, the compound is a PERK modulator.

According to some of any of the embodiments described herein, the compound is an activator of (e.g., is capable of activating) any one of XBP1, ATF6 and PERK. In some of any of the embodiments described herein, the compound is a PERK activator. According to some of any of the embodiments described herein, the compound is a PERK activator, or a compound capable of upregulating PERK activity.

Herein and in the art, the term “PERK” refers to a protein also known as “PKR-like endoplasmic reticulum kinase” and “eIF2αK3” (eIF2α kinase 3).

An exemplary activity of PERK (which may be upregulated according to some embodiments described herein) is phosphorylation of eIF2α.

Herein, the phrase “upregulating PERK activity” means increasing an activity of PERK and/or the processes and/or protein activities which result in the production of PERK. Upregulation may optionally be effected on the genomic and/or the transcript level by promoting transcription and/or translation of one or more proteins involved in the activity and/or production of PERK; and/or on the protein level by activating one or more proteins involved in increasing the activity of PERK (e.g., by stabilizing a protein to prevent its degradation, phosphorylation/dephosphorylation, increasing UPR as discussed herein, and the like).

In some of any of the embodiments described herein, increasing or upregulating PERK activity is by at least 5%, or by at least 10%, or by at least 20%, or by at least 25%, or by at least 30%, or by at least 40%, or by at least 50%, or by at least 60%, or by at least 70%, or by at least 80%, or by at least 90%, or by at least 100% (2-folds), or by more, for example, by 200% (3-folds), 300% (4-folds), 400% (5-folds) or even 1,000% (10-folds) or higher, relative to the PERK activity in the cell without a compound as defined herein.

According to an aspect of some embodiments of the present invention there is provided a compound for use in treating a viral infection, wherein the compound is a PERK activator or a compound capable of upregulating PERK activity.

According to an aspect of some embodiments of the present invention, there is provided a method of treating a viral infection in a subject in need thereof, the method comprising administering to the subject a therapeutically affective amount of a PERK activator or a compound capable of upregulating PERK activity.

As used herein, the phrase “therapeutically effective amount” describes a dose of an active ingredient (a compound capable of upregulating PERK activity as described herein) or a composition comprising the active ingredient that will provide the therapeutic effect for which the active ingredient is indicated (for example, upregulating an activity of PERK and/or treating a viral infection), optionally by relieving to some extent one or more of the symptoms of a condition being treated (e.g., according to any of the respective embodiments described herein).

Herein, the term “treating” refers to inhibiting, preventing or arresting the development of a pathology (herein, a viral infection) and/or causing the reduction, remission, or regression of a pathology (a viral infection). Those of skill in the art will understand that various methodologies and assays can be used to assess the development of a viral infection, and similarly, various methodologies and assays may be used to assess the reduction, remission or regression of a viral infection.

According to some embodiments, treating a viral infection comprises reducing a load of virus in the subject.

By “reducing a load” it is meant reducing a population of a virus that causes the viral infection, by, for example, killing and/or inhibiting growth of the virus.

As used herein, the term “subject” includes mammals, preferably human beings at any age which suffer from the pathology. Preferably, this term encompasses individuals who are at risk to develop the pathology.

A viral infection according to any of the embodiments described herein may be associated with any virus species and/or strain.

Herein, the term “virus” refers to an agent that replicates only inside living cells of an organism, and encompasses agents composed solely of a nucleic acid, such as viroids. Examples of viruses include, without limitation, double strand DNA viruses, such as adenoviruses, herpesviruses (e.g., varicella zoster virus, herpes simplex virus-1 and/or herpes simplex virus-2), polyomaviruses (e.g., JC virus), and poxviruses; single strand DNA viruses, such as parvoviruses; double strand RNA viruses, such as reoviruses (e.g., epizootic hemorrhagic disease virus); (+)-single strand RNA viruses, such as coronaviruses (e.g., coronavirus HKU1, coronavirus NL63, coronavirus 229E, coronavirus OC43, Middle East respiratory syndrome coronavirus (MERS-CoV) and/or SARS-CoV), flaviviruses (e.g., hepatitis C virus and/or West Nile virus), hepeviruses (e.g., hepatitis E virus), picornaviruses (e.g., hepatitis A virus, enteroviruses and/or rhinoviruses, such as human enteroviruses and/or rhinoviruses) and togaviruses; (−)-single strand RNA viruses, such as orthomyxoviruses (e.g., influenza A virus and/or influenza B virus), filoviruses (e.g., Ebola virus), paramyxoviruses (e.g., parainfluenza virus type 1, 2, 3 and/or 4), pneumoviruses (e.g., respiratory syncytial virus and/or human metapneumovirus) and rhabdoviruses (e.g., vesicular stomatitis Indiana virus); RNA retroviruses; DNA retroviruses, such as hepadnaviruses (e.g., hepatitis B virus); satellite viruses, such as deltaviruses (e.g., hepatitis D virus); and viroids.

According to some of any of the embodiments described herein, the viral infection is associated with double strand DNA viruses, single strand DNA viruses, double strand RNA viruses, (+)-single strand RNA viruses, (−)-single strand RNA viruses, RNA retroviruses; DNA retroviruses, satellite viruses, and/or viroids.

Exemplary viruses that cause disease include, but are not limited to, those set forth in Table A hereinbelow.

TABLE A

| | Baltimore | | |
|---|---|---|---|
| Family | group | Important species | Envelopment |
| Adenoviridae | Group I | Adenovirus | non-enveloped |
| | (dsDNA) | | |
| Herpesviridae | Group I | Herpes simplex, | enveloped |
| | (dsDNA) | type 1, Herpes | |
| | | simplex, type 2, | |
| | | Varicella-zoster virus, | |
| | | Epstein-Barr | |
| | | virus, Human | |
| | | cytomegalovirus, | |
| | | Human herpesvirus, | |
| | | type 8 | |
| Papillomaviridae | Group I | Human papillomavirus | non-enveloped |
| | (dsDNA) | | |
| Polyomaviridae | Group I | BK virus, JC virus | non-enveloped |
| | (dsDNA) | | |
| Poxviridae | Group I | Smallpox | enveloped |
| | (dsDNA) | | |
| Hepadnaviridae | Group VII | Hepatitis B virus | enveloped |
| | (dsDNA-RT) | | |
| Parvoviridae | Group II | Parvovirus B19 | non-enveloped |
| | (ssDNA) | | |
| Astroviridae | Group IV | Human astrovirus | non-enveloped |
| | (positive-sense | | |
| | ssRNA) | | |
| Caliciviridae | Group IV | Norwalk virus | non-enveloped |
| | (positive-sense | | |
| | ssRNA) | | |
| Picornaviridae | Group IV | coxsackievirus, | non-enveloped |
| | (positive-sense | hepatitis A virus, | |
| | ssRNA) | poliovirus, rhinovirus | |
| Coronaviridae | Group IV | Severe acute | enveloped |
| | (positive-sense | respiratory syndrome | |
| | ssRNA) | virus | |
| Flaviviridae | Group IV | Hepatitis C virus, | enveloped |
| | (positive- | yellow fever virus, | |
| | sense | dengue virus, West | |
| | ssRNA) | Nile virus, TBE | |
| | | virus | |
| Togaviridae | Group IV | Rubella virus | enveloped |
| | (positive-sense | | |
| | ssRNA) | | |
| Hepeviridae | Group IV | Hepatitis E virus | non-enveloped |
| | (positive-sense | | |
| | ssRNA) | | |
| Retroviridae | Group VI | Human | enveloped |
| | (ssRNA-RT) | immunodeficiency | |
| | | virus (HIV) | |
| Orthomyxoviridae | Group V | Influenza virus | enveloped |
| | (negative- | | |
| | sense ssRNA) | | |
| Arenaviridae | Group V | Lassa virus | enveloped |
| | (negative- | | |
| | sense ssRNA) | | |
| Bunyaviridae | Group V | Crimean-Congo | enveloped |
| | (negative- | hemorrhagic fever | |
| | sense ssRNA) | virus, Hantaan | |
| | | virus | |
| Filoviridae | Group V | Ebola virus, | enveloped |
| | (negative- | Marburg virus | |
| | sense ssRNA) | | |
| Paramyxoviridae | Group V | Measles virus, | enveloped |
| | (negative- | Mumps virus, | |
| | sense ssRNA) | Parainfluenza | |
| | | virus, Respiratory | |
| | | syncytial virus, | |
| Rhabdoviridae | Group V | Rabies virus | enveloped |
| | (negative- | | |
| | sense ssRNA) | | |
| Unassigned | Group V | Hepatitis D | enveloped |
| | (negative- | | |
| | sense ssRNA) | | |
| Reoviridae | Group III | Rotavirus, | non-enveloped |
| | (dsRNA) | Orbivirus, | |
| | | Coltivirus, Banna | |
| | | virus | |

According to some of any of the embodiments described herein, the virus is an RNA virus.

According to some of any of the embodiments described herein, the virus is coronavirus, rhabdovirus and/or reovirus.

According to specific embodiments, the virus is a coronavirus.

According to specific embodiments, a clinical manifestation of Coronavirus infection includes symptoms selected from the group consisting of inflammation in the lung, alveolar damage, fever, cough, shortness of breath, diarrhea, organ failure, pneumonia and/or septic shock.

As used herein, “Coronavirus” refers to enveloped positive-stranded RNA viruses that belong to the family Coronaviridae and the order Nidovirales.

The coronavirus according to any of the respective embodiments described herein is optionally a betacoronavirus, for example, an embecovirus (a.k.a. lineage A), sarbecovirus (a.k.a. lineage B), merbecovirus (a.k.a. lineage C), nobecovirus (a.k.a. lineage D), and hibecovirus. Exemplary betacoronaviruses include SARS-related coronavirus (a species of sarbecovirus), human coronavirus OC43, and human coronavirus HKU1, including any strains thereof (e.g., SARS-CoV-2).

Alternatively or additionally, examples of coronaviruses which are contemplated herein include, but are not limited to, 229E, NL63, OC43, and HKU1 with the first two classified as antigenic group 1 and the latter two belonging to group 2, typically leading to an upper respiratory tract infection manifested by common cold symptoms.

However, Coronaviruses, which are zoonotic in origin, can evolve into a strain that can infect human beings leading to fatal illness. Thus particular examples of Coronaviruses contemplated herein are SARS-CoV, Middle East respiratory syndrome Coronavirus (MERS-CoV), and the recently identified SAR-CoV-2 [causing 2019-nCoV (also referred to as “COVID-19”)].

It would be appreciated that any Coronavirus strain is contemplated herein even though SAR-CoV-2 is emphasized in a detailed manner.

According to specific embodiments, the virus is a Rhabdovirus.

A clinical manifestation of Rhabdovirus infection includes symptoms selected from the group consisting of fever, headache, muscle weakness, malaise, nausea, vomiting, diarrhea, abdominal pain, photophobia, confusion, seizures, and paralysis.

As used herein, “Rhabdovirus” refers to enveloped negative-stranded RNA viruses that belong to the family Rhabdoviridae and the order Mononegavirales.

The Rhabdovirus according to any of the respective embodiments described herein is optionally a member of the genus Lyssavirus or Vesiculovirus, for example, rabies lyssavirus or Vesicular stomatitis Indiana virus (VSV). Additional Rhabdoviruses include Chandipura virus and Mokola virus, including any strains thereof.

It would be appreciated that any Rhabdovirus strain is contemplated herein even though VSV is emphasized in a detailed manner.

According to some of any of the embodiments described herein, the virus is a severe acute respiratory syndrome coronavirus (SARS-CoV) or a Vesicular stomatitis Indiana virus (VSV).

In some embodiments, the viral infection treatable using a compound as described herein in any of the respective embodiments is associated with a virus other than a coronavirus or a vesicular stomatitis Indiana virus (VSV). Examples of other viruses include, but are not limited to, CMV (cytomegalovirus), HRV (human rhinoviruses), hepatovirus A, HMV (human meningo virus), and HIV (human immunodeficiency virus).

According to some of any of the embodiments described herein, the treatment results in inhibition of exit of a viral glycoprotein from endoplasmic reticulum.

According to an aspect of some embodiments of the present invention, there is provided a method of reducing a population of a virus, the method comprising contacting the virus (e.g., in vivo, ex-vivo or in vitro) with a PERK activator or a compound capable of upregulating PERK activity.

According to an aspect of some embodiments of the present invention, there is provided a use of a PERK activator or a compound capable of upregulating PERK activity in treating a viral infection in a subject in need thereof and/or in the manufacturing of a medicament for treating a viral infection in a subject in need thereof and/or in reducing a population of a virus, as described herein.

Herein, the phrase “treating a viral infection” means reducing and/or alleviating symptoms of a viral infection, inhibiting growth and/or killing the virus, reducing a population of the virus, and the like. Treating a viral infection further refers to the amelioration of any biological or pathological endpoints that is mediated in part by the presence of the virus in the subject, and whose outcome can be affected by reducing the level of viral gene products present.

Any PERK activator is contemplated in the context of the present embodiments.

Examples of commercially available PERK activators include, but are not limited to, DHBDC (Calbiochem®, Sigma-Aldrich™), EIF2AK3 Activator, CCT020312 (Calbiochem®, Sigma-Aldrich™), guanabenz, thapsigargin, bortezomib, and Integrated Stress Response Inhibitor (ISRIB).

According to some of any of the embodiments described herein, PERK activators usable in the context of the methods and uses described herein can be collectively represented by Formula I:

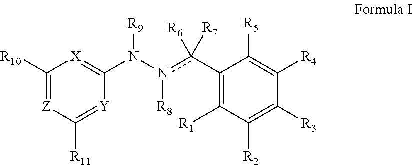

wherein:
the dashed line denotes a saturated or unsaturated bond;
X is N or CR₁₂;
Y is N or CR₁₃;
Z is N or CR₁₄;
R₁-R₇ and R₁₀-R₁₄ are each independently hydrogen, alkyl, alkenyl, alkynyl, cycloalkyl, aryl, heteroaryl, heteroalicyclic, halo, hydroxy, alkoxy, aryloxy, thiohydroxy, thioalkoxy, thioaryloxy, sulfinyl, sulfonyl, sulfonate, sulfate, cyano, nitro, azide, phosphonyl, phosphinyl, carbonyl, thiocarbonyl, a urea group, a thiourea group, O-carbamyl, N-carbamyl, O-thiocarbamyl, N-thiocarbamyl, S-thiocarbamyl, C-amido, N-amido, C-carboxy, O-carboxy, sulfonamido, guanyl, guanidinyl, hydrazine, hydrazide, thiohydrazide, and/or amino; and
R₈ and R₉ are each independently hydrogen or alkyl,
wherein when the dashed line denotes an unsaturated bond, R₇ and R₈ are absent (see, Formula II).

According to some of any of the embodiments described herein for any of the Formulae described herein, R₁ is hydrogen or hydroxy.

According to some of any of the embodiments described herein for any of the Formulae described herein, R₁ is hydrogen.

According to some of any of the embodiments described herein for any of the Formulae described herein, R₂-R₄ are each independently hydrogen, hydroxy, alkoxy, and/or alkyl.

According to some of any of the embodiments described herein for any of the Formulae described herein, at least one, at least two, or all, of R₂-R₄ is hydroxy.

According to some of any of the embodiments described herein for any of the Formulae described herein, R₁ is hydrogen and at least one of R₂-R₄ is hydroxy. According to some of any of the embodiments described herein for any of the Formulae described herein, R₂ and R₃ are each hydroxy.

According to some of any of the embodiments described herein for any of the Formulae described herein, R₁ is hydrogen and at least two of R₂-R₄ is hydroxy.

According to some of any of the embodiments described herein for any of the Formulae described herein, R₁ is hydrogen and R₂ and R₃ are each hydroxy.

According to some of any of the embodiments described herein for any of the Formulae described herein, at least one, at least two, or all, of R₂-R₄ is alkoxy.

According to some of any of the embodiments described herein for any of the Formulae described herein, R₁ is hydrogen and at least one of R₂-R₄ is alkoxy.

According to some of any of the embodiments described herein for any of the Formulae described herein, at least one, at least two, or all, of R₂-R₄ is trifluoromethyl.

According to some of any of the embodiments described herein for any of the Formulae described herein, R₁ is hydrogen and at least one, at least two, or all, of R₂-R₄ is trifluoromethyl.

According to some of any of the embodiments described herein for any of the Formulae described herein, R₅ is hydrogen.

According to some of any of the embodiments described herein for any of the Formulae described herein, R₆ is hydrogen.

According to some of any of the embodiments described herein for any of the Formulae described herein, R₅ and R₆ are each hydrogen.

According to some of any of the embodiments described herein for any of the Formulae described herein, R₁, R₅ and R₆ are each hydrogen.

According to some of any of the embodiments described herein for any of the Formulae described herein, R₁, R₅ and R₆ are each hydrogen, and at least one, at least two, or all, of R₂-R₄ is/are selected from hydroxy, alkoxy and trifluoromethyl.

According to some of any of the embodiments described herein for any of the Formulae described herein, R₁, R₅ and R₆ are each hydrogen, and at least one, at least two, or all, of R₂-R₄ is/are hydroxy.

According to some of any of the embodiments described herein for any of the Formulae described herein, R₁, R₅ and R₆ are each hydrogen, and R₂ and R₃ are each hydroxy.

According to some of any of the embodiments described herein for any of the Formulae described herein, R₉ is hydrogen or methyl.

According to some of any of the embodiments described herein for any of the Formulae described herein, R₉ is hydrogen.

According to some of any of the embodiments described herein for any of the Formulae described herein, R₉ is methyl.

According to some of any of the embodiments described herein for any of the Formulae described herein, the dashed line denotes an unsaturated bond.

According to some of any of the embodiments described herein for any of the Formulae described herein, the dashed line denotes a saturated bond and R₇ and R₈ are each hydrogen (see, Formula IIa), or the dashed line denotes an unsaturated bond and R₇ and R₈ are each absent (see, Formula II).

According to some of any of the embodiments described herein, the compound is represented by Formula II:

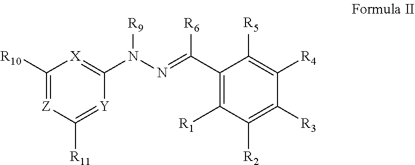

or a pharmaceutical acceptable salt thereof,
wherein R₁-R₆, R₉-R₁₄, X, Y and Z are as described herein in any of the respective embodiments.

According to some of any of the embodiments described herein, the compound is represented by Formula IIa:

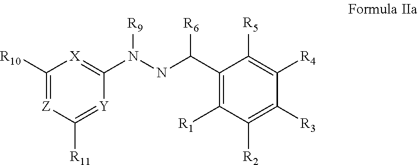

or a pharmaceutical acceptable salt thereof,
wherein R₁-R₆, R₉-R₁₄, X, Y and Z are as described herein in any of the respective embodiments.

According to some of any of the embodiments described herein for any of the Formulae described herein, R₈ and R₉ are each independently hydrogen or alkyl.

According to some of any of the embodiments described herein for any of the Formulae described herein, R₈ is absent and R₉ is hydrogen or alkyl.

According to some of any of the embodiments described herein for any of the Formulae described herein, R₉ is hydrogen.

According to some of any of the embodiments described herein for any of the Formulae described herein, R₉ is alkyl.

According to some of any of the embodiments described herein for any of the Formulae described herein, R₉ is methyl.

According to some of any of the embodiments described herein for any of the Formulae described herein, at least one, or at least two, or all, of X, Y and Z is nitrogen; and R₉ is hydrogen or alkyl.

According to some of any of the embodiments described herein for any of the Formulae described herein, at least one, or at least two, or all, of X, Y and Z is nitrogen; and R₉ is hydrogen or methyl.

According to some of any of the embodiments described herein for any of the Formulae described herein, X and Y are each nitrogen, Z is CH, and R₉ is hydrogen or alkyl.

According to some of any of the embodiments described herein for any of the Formulae described herein, X and Y are each nitrogen, Z is CH, and R₉ is hydrogen or methyl.

According to some of any of the embodiments described herein for any of the Formulae described herein, R₁, R₅ and R₆ are each hydrogen; at least one, at least two, or all, of R₂-R₄ is/are selected from hydroxy, alkoxy and trifluoromethyl; at least one, or at least two, or all, of X, Y and Z is nitrogen; and R₉ is hydrogen or alkyl.

According to some of any of the embodiments described herein for any of the Formulae described herein, R₁, R₅ and R₆ are each hydrogen; at least one, at least two, or all, of R₂-R₄ is/are selected from hydroxy, alkoxy and trifluoromethyl; at least one, or at least two, or all, of X, Y and Z is nitrogen; and R₉ is hydrogen or methyl.

According to some of any of the embodiments described herein for any of the Formulae described herein, R₁, R₅ and R₆ are each hydrogen; at least one, at least two, or all, of R₂-R₄ is/are selected from hydroxy, alkoxy and trifluoromethyl; X and Y are each nitrogen, Z is CH, and R₉ is hydrogen or alkyl.

According to some of any of the embodiments described herein for any of the Formulae described herein, R₁, R₅ and R₆ are each hydrogen; at least one, at least two, or all, of R₂-R₄ is/are selected from hydroxy, alkoxy and trifluoromethyl; X and Y are each nitrogen, Z is CH, and R₉ is hydrogen or methyl.

According to some of any of the embodiments described herein for any of the Formulae described herein, R₁, R₅ and R₆ are each hydrogen; R₂ and R₃ are each hydroxy; at least one, or at least two, or all, of X, Y and Z is nitrogen; and R₉ is hydrogen or alkyl.

According to some of any of the embodiments described herein for any of the Formulae described herein, R₁, R₅ and R₆ are each hydrogen; R₂ and R₃ are each hydroxy; at least one, or at least two, or all, of X, Y and Z is nitrogen; and R₉ is hydrogen or methyl.

According to some of any of the embodiments described herein for any of the Formulae described herein, R₁, R₅ and R₆ are each hydrogen; R₂ and R₃ are each hydroxy; X and Y are each nitrogen, Z is CH, and R₉ is hydrogen or alkyl.

According to some of any of the embodiments described herein for any of the Formulae described herein, R₁, R₅ and R₆ are each hydrogen; R₂ and R₃ are each hydroxy; X and Y are each nitrogen, Z is CH, and R₉ is hydrogen or methyl.

According to some of any of the embodiments described herein for any of the Formulae described herein, R₉ is alkyl. In some embodiments, the alkyl is unsubstituted. In some embodiments, R₉ is C₁₋₄-alkyl, optionally unsubstituted C₁₋₄-alkyl. In exemplary embodiments, R₉ is methyl.

According to some of any of the embodiments described herein for any of the Formulae described herein, R₁₂-R₁₄ are each hydrogen.

According to some of any of the embodiments described herein for any of the Formulae described herein, at least one, or at least two, or all, of X, Y and Z is nitrogen. According to some of any of the embodiments described herein for any of the Formulae described herein, X is nitrogen.

According to some of any of the embodiments described herein for any of the Formulae described herein, Y is nitrogen.

According to some of any of the embodiments described herein for any of the Formulae described herein, X and Y are each nitrogen.

According to some of any of the embodiments described herein for any of the Formulae described herein, Z is CH.

According to some of any of the embodiments described herein for any of the Formulae described herein, X and Y are each nitrogen, and Z is CH.

According to some of any of the embodiments described herein for any of the Formulae described herein, X and Y are each nitrogen, Z is CH, the dashed line denotes an unsaturated bond and R₇ and R₈ are each absent (see, Formula III).

According to some of any of the embodiments described herein, the compound is represented by Formula III:

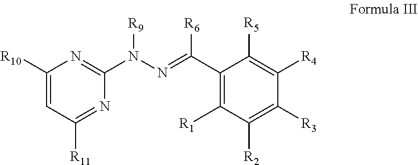

or a pharmaceutical acceptable salt thereof,
wherein R₁-R₆ and R₉-R₁₁ are as described herein in any of the respective embodiments.

According to some of any of the embodiments described herein for any of the Formulae described herein, R₁, R₅ and R₆ are each hydrogen; at least one, at least two, or all, of R₂-R₄ is/are selected from hydroxy, alkoxy and trifluoromethyl; and at least one, or at least two, or all, of X, Y and Z is nitrogen.

According to some of any of the embodiments described herein for any of the Formulae described herein, R₁, R₅ and R₆ are each hydrogen; at least one, at least two, or all, of R₂-R₄ is/are selected from hydroxy, alkoxy and trifluoromethyl; X and Y are each nitrogen, and Z is CH.

According to some of any of the embodiments described herein for any of the Formulae described herein, R₁, R₅ and R₆ are each hydrogen; at least one, at least two, or all, of R₂-R₄ is/are selected from hydroxy, alkoxy and trifluoromethyl; X and Y are each nitrogen, Z is CH, the dashed line denotes an unsaturated bond and R₇ and R₈ are each absent.

According to some of any of the embodiments described herein for any of the Formulae described herein, R₁, R₅ and R₆ are each hydrogen; R₂ and R₃ are each hydroxy; and at least one, or at least two, or all, of X, Y and Z is nitrogen.

According to some of any of the embodiments described herein for any of the Formulae described herein, R₁, R₅ and R₆ are each hydrogen; R₂ and R₃ are each hydroxy; X and Y are each nitrogen, and Z is CH.

According to some of any of the embodiments described herein for any of the Formulae described herein, R₁, R₅ and R₆ are each hydrogen; R₂ and R₃ are each hydroxy; X and Y are each nitrogen, Z is CH, the dashed line denotes an unsaturated bond and R₇ and R₈ are each absent.

According to some of any of the embodiments described herein for any of the Formulae described herein, R₁, R₅ and R₆ are each hydrogen; R₂ and R₃ are each hydroxy; at least one, or at least two, or all, of X, Y and Z is nitrogen, and R₉ is hydrogen or alkyl.

According to some of any of the embodiments described herein for any of the Formulae described herein, R₁, R₅ and R₆ are each hydrogen; R₂ and R₃ are each hydroxy; X and Y are each nitrogen, Z is CH, and R₉ is hydrogen or alkyl.

According to some of any of the embodiments described herein for any of the Formulae described herein, R₁, R₅ and R₆ are each hydrogen; R₂ and R₃ are each hydroxy; X and Y are each nitrogen, Z is CH, the dashed line denotes an unsaturated bond, R₇ and R₈ are each absent, and R₉ is hydrogen or alkyl.

According to some of any of the embodiments described herein for any of the Formulae described herein, R₁₀ and R₁₁ are each independently hydrogen, alkyl, cycloalkyl, aryl, heteroaryl, and/or heteroalicyclic, wherein each of the cycloalkyl, aryl, heteroaryl, or heteroalicyclic can be substituted, as defined herein, or non-substituted.

According to some of any of the embodiments described herein for any of the Formulae described herein, R₁₀ and R₁₁ are each independently a substituted or non-substituted phenyl.

According to some of any of the embodiments described herein for any of the Formulae described herein, R₁₀ and R₁₁ are each independently a substituted or non-substituted phenyl; R₁, R₅ and R₆ are each hydrogen; R₂-R₄ is/are selected from hydroxy, alkoxy and trifluoromethyl; and at least one, or at least two, or all, of X, Y and Z is nitrogen.

According to some of any of the embodiments described herein for any of the Formulae described herein, R₁₀ and R₁₁ are each independently a substituted or non-substituted phenyl; R₁, R₅ and R₆ are each hydrogen; R₂-R₄ is/are selected from hydroxy, alkoxy and trifluoromethyl; X and Y are each nitrogen, and Z is CH.

According to some of any of the embodiments described herein for any of the Formulae described herein, R₁₀ and R₁₁ are each independently a substituted or non-substituted phenyl; R₁, R₅ and R₆ are each hydrogen; R₂-R₄ is/are selected from hydroxy, alkoxy and trifluoromethyl; X and Y are each nitrogen, Z is CH, the dashed line denotes an unsaturated bond and R₇ and R₈ are each absent.

According to some of any of the embodiments described herein for any of the Formulae described herein, R₁₀ and R₁₁ are each independently a substituted or non-substituted phenyl; R₁, R₅ and R₆ are each hydrogen; R₂ and R₃ are each hydroxy; and at least one, or at least two, or all, of X, Y and Z is nitrogen.

According to some of any of the embodiments described herein for any of the Formulae described herein, R₁₀ and R₁₁ are each independently a substituted or non-substituted phenyl; R₁, R₅ and R₆ are each hydrogen; R₂ and R₃ are each hydroxy; X and Y are each nitrogen, and Z is CH.

According to some of any of the embodiments described herein for any of the Formulae described herein, R₁₀ and R₁₁ are each independently a substituted or non-substituted phenyl; R₁, R₅ and R₆ are each hydrogen; R₂ and R₃ are each hydroxy; X and Y are each nitrogen, Z is CH, the dashed line denotes an unsaturated bond and R₇ and R₈ are each absent (see, Formula IV-a).

According to some of any of the embodiments described herein for any of the Formulae described herein, R₁₀ and R₁₁ are each independently a substituted or non-substituted phenyl; R₁, R₅ and R₆ are each hydrogen; R₂ and R₃ are each hydroxy; X and Y are each nitrogen, Z is CH, the dashed line denotes an unsaturated bond, R₇ and R₈ are each absent; and R₉ is hydrogen or alkyl.

According to some of any of the embodiments described herein for any of the Formulae described herein, R₁₀ and R₁₁ are each independently a substituted or non-substituted phenyl; X and Y are each nitrogen, Z is CH, the dashed line denotes an unsaturated bond and R₇ and R₈ are each absent (see, Formula IV).

According to some of any of the embodiments described herein, the compound is represented by Formula IV:

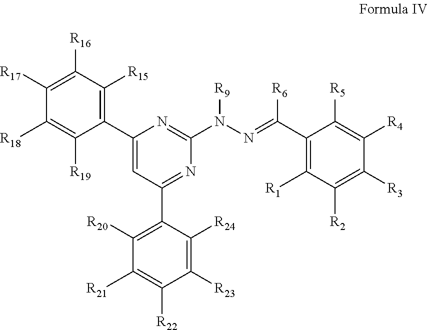

or a pharmaceutical acceptable salt thereof,
wherein R₁-R₆, R₉ and R₁₅-R₂₄ are as described herein in any of the respective embodiments.

According to some of any of the embodiments described herein, the compound is represented by Formula IV-a:

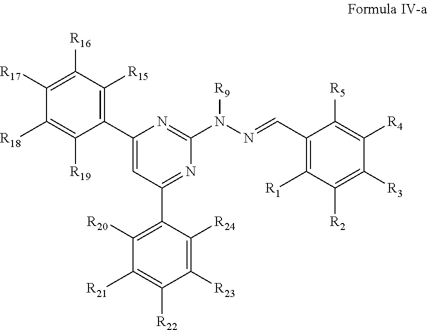

or a pharmaceutical acceptable salt thereof,
wherein R₁-R₅, R₉ and R₁₅-R₂₄ are as described herein in any of the respective embodiments.

In some embodiments of any of the embodiments described herein, R₁₇ is other than bromo. In some such embodiments, R₁₇ is other than halo.

It is to be understood that limitations regarding any of the variables R₁₅-R₂₄ apply also to other variables which are at equivalent positions in Formula IV. For example, as R₁₇ and R₂₂ are at equivalent positions in Formula IV, the limitation “R₁₇ is other than bromo” (or halo) is to be understood as meaning that “neither R₁₇ nor R₂₂ is bromo” (or halo), and vice versa; and “R₁₇ is hydrogen” is to be understood as meaning that “R₁₇ and R₂₂ are each hydrogen”, and vice versa.

In some embodiments of any of the embodiments described herein, when R₂ is alkoxy, neither R₁₇ nor R₂₂ is bromo. In some such embodiments, neither R₁₇ nor R₂₂ is halo.

In some embodiments of any of the embodiments described herein, when R₂ is methoxy, neither R₁₇ nor R₂₂ is bromo. In some such embodiments, neither R₁₇ nor R₂₂ is halo.

In some embodiments of any of the embodiments described herein, when R₂ is methoxy, R₉ is hydrogen or alkyl, and R₁₅, R₁₆, R₁₈-R₂₁ are each hydrogen, neither R₁₇ nor R₂₂ is bromo. In some such embodiments, neither R₁₇ nor R₂₂ is halo.

In some embodiments of any of the embodiments described herein, when R₂ is methoxy, R₉ is methyl, and R₁₅, R₁₆, R₁₈-R₂₁ are each hydrogen, neither R₁₇ nor R₂₂ is bromo. In some such embodiments, neither R₁₇ nor R₂₂ is halo. In some of any of the embodiments described herein wherein R₁₇ (and/or R₂₂) is other than bromo, R₁₇ (and/or R₂₂) is a halo other than bromo, for example, chloro or fluoro (e.g., halogen substituents less bulky than bromo).

In some embodiments of any of the embodiments described herein, R₁₇ is hydrogen. In some embodiments, R₂ is alkoxy, and R₁₇ is hydrogen. In some embodiments, R₂ is alkoxy, and R₁₅-R₂₄ are each hydrogen.

In some embodiments of any of the embodiments described herein, R₁₅-R₂₄ are each hydrogen (i.e., R₁₀ and R₁₁ are non-substituted phenyl). In some embodiments of any of the embodiments described herein, R₁₅-R₂₄ are each hydrogen.

According to some of any of the embodiments described herein for any of the Formulae described herein, R₁, R₅ and R₆ are each hydrogen; at least one, at least two, or all, of R₂-R₄ is/are selected from hydroxy, alkoxy and trifluoromethyl; and R₁₀ and R₁₁ are each independently a non-substituted phenyl; and at least one, or at least two, or all, of X, Y and Z is nitrogen.

According to some of any of the embodiments described herein for any of the Formulae described herein, R₁, R₅ and R₆ are each hydrogen; at least one, at least two, or all, of R₂-R₄ is/are selected from hydroxy, alkoxy and trifluoromethyl; and R₁₀ and R₁₁ are each independently a non-substituted phenyl; X and Y are each nitrogen, and Z is CH.

According to some of any of the embodiments described herein for any of the Formulae described herein, R₁, R₅ and R₆ are each hydrogen; at least one, at least two, or all, of R₂-R₄ is/are selected from hydroxy, alkoxy and trifluoromethyl; and R₁₀ and R₁₁ are each independently a non-substituted phenyl; X and Y are each nitrogen, Z is CH, the dashed line denotes an unsaturated bond and R₇ and R₈ are each absent.

According to exemplary embodiments, one or both of R₁₀ and R₁₁ is a non-substituted phenyl.

When substituted, the phenyl can be substituted by one or more of the substituents as defined herein.

According to some of any of the embodiments described herein for any of the Formulae described herein, R₁, R₅ and R₆ are each hydrogen; at least one, at least two, or all, of R₂-R₄ is/are selected from hydroxy, alkoxy and trifluoromethyl; and R₁₀ and R₁₁ are each independently a substituted or non-substituted phenyl.

According to some of any of the embodiments described herein for any of the Formulae described herein, R₁, R₅ and R₆ are each hydrogen; at least one, at least two, or all, of R₂-R₄ is/are selected from hydroxy, alkoxy and trifluoromethyl; and R₁₀ and R₁₁ are each independently a substituted or non-substituted phenyl; and at least one, or at least two, or all, of X, Y and Z is nitrogen.

According to some of any of the embodiments described herein for any of the Formulae described herein, R₁, R₅ and R₆ are each hydrogen; at least one, at least two, or all, of R₂-R₄ is/are selected from hydroxy, alkoxy and trifluoromethyl; and R₁₀ and R₁₁ are each independently a substituted or non-substituted phenyl; X and Y are each nitrogen, and Z is CH.

According to some of any of the embodiments described herein for any of the Formulae described herein, R₁, R₅ and R₆ are each hydrogen; at least one, at least two, or all, of R₂-R₄ is/are selected from hydroxy, alkoxy and trifluoromethyl; and R₁₀ and R₁₁ are each independently a substituted or non-substituted phenyl; X and Y are each nitrogen, Z is CH, the dashed line denotes an unsaturated bond and R₇ and R₈ are each absent.

According to some of any of the embodiments described herein for any of the Formulae described herein, R₁, R₅ and R₆ are each hydrogen; R₂ and R₃ are each hydroxy; and R₁₀ and R₁₁ are each independently a substituted or non-substituted phenyl.

According to some of any of the embodiments described herein, the compound is 4-((2-(4,6-diphenylpyrimidin-2-yl)-2-methylhydrazineylidene)methyl) benzene-1,2-diol, which is also referred to herein as MK-28, or a pharmaceutical acceptable salt thereof.

In some of any of the embodiments described herein, unless otherwise indicated, “MK-28” as described herein encompasses the (E)- and (Z)-4-((2-(4,6-diphenylpyrimidin-2-yl)-2-methylhydrazineylidene)methyl) benzene-1,2-diol:

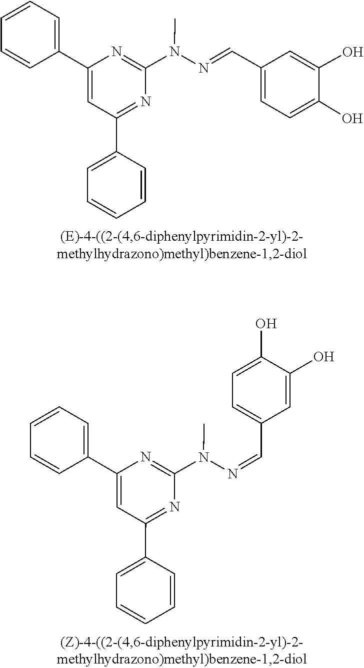 [SMILES: CN(/N=C/c1ccc(O)c(O)c1)c1nc(-c2ccccc2)cc(-c2ccccc2)n1 ; CN(/N=C\c1ccc(O)c(O)c1)c1nc(-c2ccccc2)cc(-c2ccccc2)n1]

According to some of any of the embodiments described herein, the compound is 4-((2-(4,6-diphenylpyrimidin-2-yl)hydrazono)methyl)benzene-1,2-diol, which is also referred to herein as GLB-7, or a pharmaceutical acceptable salt thereof.

In some of any of the embodiments described herein, “GLB-7” as described herein encompasses, unless otherwise indicated, the (E)- and (Z)-4-((2-(4,6-diphenylpyrimidin-2-yl)hydrazono)methyl)benzene-1,2-diol:

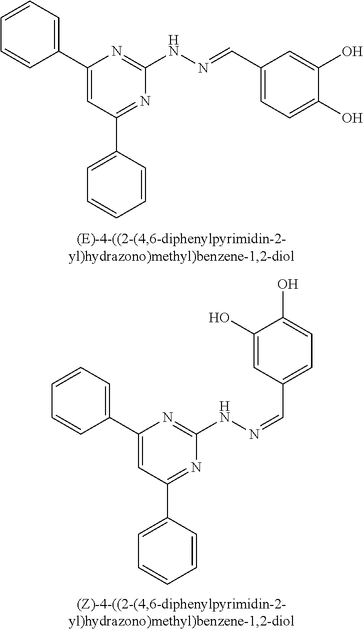 [SMILES: Oc1ccc(/C=N/Nc2nc(-c3ccccc3)cc(-c3ccccc3)n2)cc1O ; Oc1ccc(/C=N\Nc2nc(-c3ccccc3)cc(-c3ccccc3)n2)cc1O]

According to some of any of the embodiments described herein, the compound is selected from MK-28 and GLB-7, as described herein.

Compounds according to any of the aspects described herein, in which one or more of R₁-R₅ is OH are presented herein as an “enol” tautomer, but can undergo keto-enol tautomerization. Some embodiments of the present invention therefrom encompass also the “keto” tautomer of these compounds.

Exemplary keto-enol tautomers are presented in the following scheme for Compound MK-28.

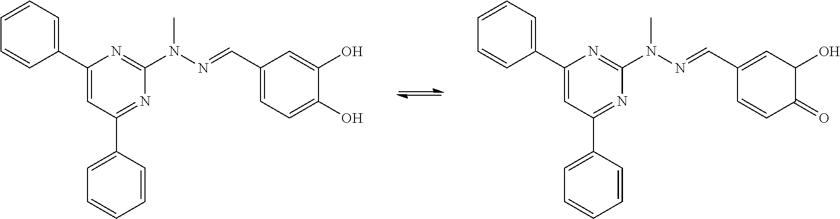 [SMILES: CN(/N=C/c1ccc(O)c(O)c1)c1nc(-c2ccccc2)cc(-c2ccccc2)n1 ; CN(/N=C/C1=CC(O)C(=O)C=C1)c1nc(-c2ccccc2)cc(-c2ccccc2)n1]

In some embodiments, compounds which present keto-enol tautomerization are in a form of the “enol” tautomer.

As used herein throughout, the term “alkyl” refers to any saturated aliphatic hydrocarbon including straight chain and branched chain groups. Preferably, the alkyl group has 1 to 20 carbon atoms. Whenever a numerical range; e.g., “1 to 20”, is stated herein, it implies that the group, in this case the hydrocarbon, may contain 1 carbon atom, 2 carbon atoms, 3 carbon atoms, etc., up to and including 20 carbon atoms. More preferably, the alkyl is a medium size alkyl having 1 to 10 carbon atoms. Most preferably, unless otherwise indicated, the alkyl is a lower alkyl having 1 to 4 carbon atoms. The alkyl group may be substituted or non-substituted. When substituted, the substituent group can be, for example, cycloalkyl, aryl, heteroaryl, heteroalicyclic, halo, hydroxy, alkoxy, aryloxy, thiohydroxy, thioalkoxy, thioaryloxy, sulfinyl, sulfonyl, sulfonate, sulfate, cyano, nitro, azide, phosphonyl, phosphinyl, oxo, imine, oxime, hydrazone, carbonyl, thiocarbonyl, a urea group, a thiourea group, O-carbamyl, N-carbamyl, O-thiocarbamyl, N-thiocarbamyl, S-thiocarbamyl, C-amido, N-amido, C-carboxy, O-carboxy, sulfonamido, guanyl, guanidinyl, hydrazine, hydrazide, thiohydrazide, and amino, as these terms are defined herein.

Herein, the term “alkenyl” describes an unsaturated aliphatic hydrocarbon comprise at least one carbon-carbon double bond, including straight chain and branched chain groups. Preferably, the alkenyl group has 2 to 20 carbon atoms. More preferably, the alkenyl is a medium size alkenyl having 2 to 10 carbon atoms. Most preferably, unless otherwise indicated, the alkenyl is a lower alkenyl having 2 to 4 carbon atoms. The alkenyl group may be substituted or non-substituted. Substituted alkenyl may have one or more substituents, whereby each substituent group can independently be, for example, alkynyl, cycloalkyl, alkynyl, aryl, heteroaryl, heteroalicyclic, halo, hydroxy, alkoxy, aryloxy, thiohydroxy, thioalkoxy, thioaryloxy, sulfinyl, sulfonyl, sulfonate, sulfate, cyano, nitro, azide, phosphonyl, phosphinyl, oxo, imine, oxime, hydrazone, carbonyl, thiocarbonyl, a urea group, a thiourea group, O-carbamyl, N-carbamyl, O-thiocarbamyl, N-thiocarbamyl, S-thiocarbamyl, C-amido, N-amido, C-carboxy, O-carboxy, sulfonamido, guanyl, guanidinyl, hydrazine, hydrazide, thiohydrazide, and amino.

Herein, the term “alkynyl” describes an unsaturated aliphatic hydrocarbon comprise at least one carbon-carbon triple bond, including straight chain and branched chain groups. Preferably, the alkynyl group has 2 to 20 carbon atoms. More preferably, the alkynyl is a medium size alkynyl having 2 to 10 carbon atoms. Most preferably, unless otherwise indicated, the alkynyl is a lower alkynyl having 2 to 4 carbon atoms. The alkynyl group may be substituted or non-substituted. Substituted alkynyl may have one or more substituents, whereby each substituent group can independently be, for example, cycloalkyl, alkenyl, aryl, heteroaryl, heteroalicyclic, halo, hydroxy, alkoxy, aryloxy, thiohydroxy, thioalkoxy, thioaryloxy, sulfinyl, sulfonyl, sulfonate, sulfate, cyano, nitro, azide, phosphonyl, phosphinyl, oxo, imine, oxime, hydrazone, carbonyl, thiocarbonyl, a urea group, a thiourea group, O-carbamyl, N-carbamyl, O-thiocarbamyl, N-thiocarbamyl, S-thiocarbamyl, C-amido, N-amido, C-carboxy, O-carboxy, sulfonamido, guanyl, guanidinyl, hydrazine, hydrazide, thiohydrazide, and amino.

A “cycloalkyl” group refers to a saturated on unsaturated all-carbon monocyclic or fused ring (i.e., rings which share an adjacent pair of carbon atoms) group wherein one of more of the rings does not have a completely conjugated pi-electron system. Examples, without limitation, of cycloalkyl groups are cyclopropane, cyclobutane, cyclopentane, cyclopentene, cyclohexane, cyclohexadiene, cycloheptane, cycloheptatriene, and adamantane. A cycloalkyl group may be substituted or non-substituted. When substituted, the substituent group can be, for example, alkyl, alkenyl, alkynyl, cycloalkyl, aryl, heteroaryl, heteroalicyclic, halo, hydroxy, alkoxy, aryloxy, thiohydroxy, thioalkoxy, thioaryloxy, sulfinyl, sulfonyl, sulfonate, sulfate, cyano, nitro, azide, phosphonyl, phosphinyl, oxo, imine, oxime, hydrazone, carbonyl, thiocarbonyl, a urea group, a thiourea group, O-carbamyl, N-carbamyl, O-thiocarbamyl, N-thiocarbamyl, S-thiocarbamyl, C-amido, N-amido, C-carboxy, O-carboxy, sulfonamido, guanyl, guanidinyl, hydrazine, hydrazide, thiohydrazide, and amino, as these terms are defined herein. When a cycloalkyl group is unsaturated, it may comprise at least one carbon-carbon double bond and/or at least one carbon-carbon triple bond.

An “aryl” group refers to an all-carbon monocyclic or fused-ring polycyclic (i.e., rings which share adjacent pairs of carbon atoms) having a completely conjugated pi-electron system. Examples, without limitation, of aryl groups are phenyl, naphthalenyl and anthracenyl. The aryl group may be substituted or non-substituted. When substituted, the substituent group can be, for example, alkyl, alkenyl, alkynyl, cycloalkyl, aryl, heteroaryl, heteroalicyclic, halo, hydroxy, alkoxy, aryloxy, thiohydroxy, thioalkoxy, thioaryloxy, sulfinyl, sulfonyl, sulfonate, sulfate, cyano, nitro, azide, phosphonyl, phosphinyl, oxo, imine, oxime, hydrazone, carbonyl, thiocarbonyl, a urea group, a thiourea group, O-carbamyl, N-carbamyl, O-thiocarbamyl, N-thiocarbamyl, S-thiocarbamyl, C-amido, N-amido, C-carboxy, O-carboxy, sulfonamido, guanyl, guanidinyl, hydrazine, hydrazide, thiohydrazide, and amino, as these terms are defined herein.

A “heteroaryl” group refers to a monocyclic or fused ring (i.e., rings which share an adjacent pair of atoms) having in the ring(s) one or more atoms, such as, for example, nitrogen, oxygen and sulfur and, in addition, having a completely conjugated pi-electron system. Examples, without limitation, of heteroaryl groups include pyrrole, furan, thiophene, imidazole, oxazole, thiazole, pyrazole, pyridine, pyrimidine, quinoline, isoquinoline and purine. The heteroaryl group may be substituted or non-substituted. When substituted, the substituent group can be, for example, alkyl, alkenyl, alkynyl, cycloalkyl, aryl, heteroaryl, heteroalicyclic, halo, hydroxy, alkoxy, aryloxy, thiohydroxy, thioalkoxy, thioaryloxy, sulfinyl, sulfonyl, sulfonate, sulfate, cyano, nitro, azide, phosphonyl, phosphinyl, oxo, imine, oxime, hydrazone, carbonyl, thiocarbonyl, a urea group, a thiourea group, O-carbamyl, N-carbamyl, O-thiocarbamyl, N-thiocarbamyl, S-thiocarbamyl, C-amido, N-amido, C-carboxy, O-carboxy, sulfonamido, guanyl, guanidinyl, hydrazine, hydrazide, thiohydrazide, and amino, as these terms are defined herein.

A “heteroalicyclic” group refers to a monocyclic or fused ring group having in the ring(s) one or more atoms such as nitrogen, oxygen and sulfur. The rings may also have one or more double bonds. However, the rings do not have a completely conjugated pi-electron system. The heteroalicyclic may be substituted or non-substituted. When substituted, the substituted group can be, for example, alkyl, alkenyl, alkynyl, cycloalkyl, aryl, heteroaryl, heteroalicyclic, halo, hydroxy, alkoxy, aryloxy, thiohydroxy, thioalkoxy, thioaryloxy, sulfinyl, sulfonyl, sulfonate, sulfate, cyano, nitro, azide, phosphonyl, phosphinyl, oxo, imine, oxime, hydrazone, carbonyl, thiocarbonyl, a urea group, a thiourea group, O-carbamyl, N-carbamyl, O-thiocarbamyl, N-thiocarbamyl, S-thiocarbamyl, C-amido, N-amido, C-carboxy, O-carboxy, sulfonamido, guanyl, guanidinyl, hydrazine, hydrazide, thiohydrazide, and amino, as these terms are defined herein. Representative examples are piperidine, piperazine, tetrahydrofuran, tetrahydropyran, morpholine and the like.

Herein, the terms “amine” and “amino” each refer to either a —NR′R″ group or a —N⁺R′R″R′″ group, wherein R′, R″ and R′″ are each hydrogen or a substituted or non-substituted alkyl, alkenyl, alkynyl, cycloalkyl, heteroalicyclic (linked to amine nitrogen via a ring carbon thereof), aryl, or heteroaryl (linked to amine nitrogen via a ring carbon thereof), as defined herein. Optionally, R′, R″ and R′″ are hydrogen or alkyl comprising 1 to 4 carbon atoms. Optionally, R′ and R″ (and R′″, if present) are hydrogen. When substituted, the carbon atom of an R′, R″ or R′″ hydrocarbon moiety which is bound to the nitrogen atom of the amine is not substituted by oxo (unless explicitly indicated otherwise), such that R′, R″ and R′″ are not (for example) carbonyl, C-carboxy or amide, as these groups are defined herein.

An “azide” group refers to a —N═N⁺═N— group.

An “alkoxy” group refers to any of an —O-alkyl, —O-alkenyl, —O-alkynyl, —O-cycloalkyl, and —O-heteroalicyclic group, as defined herein.

An “aryloxy” group refers to both an —O-aryl and an —O-heteroaryl group, as defined herein.

A “hydroxy” group refers to a —OH group.

A “thiohydroxy” or “thiol” group refers to a —SH group.

A “thioalkoxy” group refers to any of an —S-alkyl, —S-alkenyl, —S-alkynyl, —S-cycloalkyl, and —S-heteroalicyclic group, as defined herein.

A “thioaryloxy” group refers to both an —S-aryl and an —S-heteroaryl group, as defined herein.

A “carbonyl” or “acyl” group refers to a —C(═O)—R′ group, where R′ is defined as hereinabove.

A “thiocarbonyl” group refers to a —C(═S)—R′ group, where R′ is as defined herein.

A “C-carboxy” group refers to a —C(═O)—O—R′ group, where R′ is as defined herein.

An “O-carboxy” group refers to an R′C(═O)—O— group, where R′ is as defined herein.

A “carboxylic acid” group refers to a —C(═O)OH group.

An “oxo” group refers to a =O group.

An “imine” group refers to a =N—R′ group, where R′ is as defined herein.

An “oxime” group refers to a =N—OH group.

A “hydrazone” group refers to a =N—NR′R″ group, where each of R′ and R″ is as defined herein.

A “halo” group refers to fluorine, chlorine, bromine or iodine.

A “sulfinyl” group refers to an —S(═O)—R′ group, where R′ is as defined herein.

A “sulfonyl” group refers to an —S(═O)₂—R′ group, where R′ is as defined herein.

A “sulfonate” group refers to an —S(═O)₂—O—R′ group, where R′ is as defined herein.

A “sulfate” group refers to an —O—S(═O)₂—O—R′ group, where R′ is as defined as herein.

A “sulfonamide” or “sulfonamido” group encompasses both S-sulfonamido and N-sulfonamido groups, as defined herein.

An “S-sulfonamido” group refers to a —S(═O)₂—NR′R″ group, with each of R′ and R″ as defined herein.

An “N-sulfonamido” group refers to an R'S(═O)₂—NR″— group, where each of R′ and R″ is as defined herein.

An “O-carbamyl” group refers to an —OC(═O)—NR′R″ group, where each of R′ and R″ is as defined herein.

An “N-carbamyl” group refers to an R′OC(═O)—NR″— group, where each of R′ and R″ is as defined herein.

An “O-thiocarbamyl” group refers to an —OC(═S)—NR′R″ group, where each of R′ and R″ is as defined herein.

An “N-thiocarbamyl” group refers to an R′OC(═S)NR″— group, where each of R′ and R″ is as defined herein.

An “S-thiocarbamyl” group refers to an —SC(═O)—NR′R″ group, where each of R′ and R″ is as defined herein.

An “amide” or “amido” group encompasses C-amido and N-amido groups, as defined herein.

A “C-amido” group refers to a —C(═O)—NR′R″ group, where each of R′ and R″ is as defined herein.

An “N-amido” group refers to an R′C(═O)—NR″— group, where each of R′ and R″ is as defined herein.

A “urea group” refers to an —N(R′)—C(═O)—NR″R′″ group, where each of R′, R″ and R″ is as defined herein.

A “thiourea group” refers to a —N(R′)—C(═S)—NR″R′″ group, where each of R′, R″ and R″ is as defined herein.

A “nitro” group refers to an —NO₂ group.

A “cyano” group refers to a —C≡N group.

The term “phosphonyl” or “phosphonate” describes a —P(═O)(OR′)(OR″) group, with R′ and R″ as defined hereinabove.

The term “phosphate” describes an —O—P(═O)(OR′)(OR″) group, with each of R′ and R″ as defined hereinabove.

The term “phosphinyl” describes a —PR′R″ group, with each of R′ and R″ as defined hereinabove.

The term “hydrazine” describes a —NR′—NR″R′″ group, with R′, R″, and R′″ as defined herein.

As used herein, the term “hydrazide” describes a —C(═O)—NR′—NR″R′″ group, where R′, R″ and R′″ are as defined herein.

As used herein, the term “thiohydrazide” describes a —C(═S)—NR′—NR″R′″ group, where R′, R″ and R′″ are as defined herein.

A “guanidinyl” group refers to an —RaNC(═NRd)-NRbRc group, where each of Ra, Rb, Rc and Rd can be as defined herein for R′ and R″.

A “guanyl” or “guanine” group refers to an RaRbNC(═NRd)-group, where Ra, Rb and Rd are as defined herein.

For any of the embodiments described herein, the compound described herein may be in a form of a salt, for example, a pharmaceutically acceptable salt, and/or in a form of a prodrug.

As used herein, the phrase “pharmaceutically acceptable salt” refers to a charged species of the parent compound and its counter-ion, which is typically used to modify the solubility characteristics of the parent compound and/or to reduce any significant irritation to an organism by the parent compound, while not abrogating the biological activity and properties of the administered compound. A pharmaceutically acceptable salt of a compound as described herein can alternatively be formed during the synthesis of the compound, e.g., in the course of isolating the compound from a reaction mixture or re-crystallizing the compound.

In the context of some of the present embodiments, a pharmaceutically acceptable salt of the compounds described herein may optionally be an acid addition salt and/or a base addition salt.

An acid addition salt comprises at least one basic (e.g., amine and/or guanidinyl) group of the compound which is in a positively charged form (e.g., wherein the basic group is protonated), in combination with at least one counter-ion, derived from the selected acid, that forms a pharmaceutically acceptable salt. The acid addition salts of the compounds described herein may therefore be complexes formed between one or more basic groups of the compound and one or more equivalents of an acid.

A base addition salt comprises at least one acidic (e.g., carboxylic acid) group of the compound which is in a negatively charged form (e.g., wherein the acidic group is deprotonated), in combination with at least one counter-ion, derived from the selected base, that forms a pharmaceutically acceptable salt. The base addition salts of the compounds described herein may therefore be complexes formed between one or more acidic groups of the compound and one or more equivalents of a base.

Depending on the stoichiometric proportions between the charged group(s) in the compound and the counter-ion in the salt, the acid additions salts and/or base addition salts can be either mono-addition salts or poly-addition salts.

The phrase “mono-addition salt”, as used herein, refers to a salt in which the stoichiometric ratio between the counter-ion and charged form of the compound is 1:1, such that the addition salt includes one molar equivalent of the counter-ion per one molar equivalent of the compound.

The phrase “poly-addition salt”, as used herein, refers to a salt in which the stoichiometric ratio between the counter-ion and the charged form of the compound is greater than 1:1 and is, for example, 2:1, 3:1, 4:1 and so on, such that the addition salt includes two or more molar equivalents of the counter-ion per one molar equivalent of the compound.

An example, without limitation, of a pharmaceutically acceptable salt would be an ammonium cation or guanidinium cation and an acid addition salt thereof, and/or a carboxylate anion and a base addition salt thereof.

The base addition salts may include a cation counter-ion such as sodium, potassium, ammonium, calcium, magnesium and the like, that forms a pharmaceutically acceptable salt.

The acid addition salts may include a variety of organic and inorganic acids, such as, but not limited to, hydrochloric acid which affords a hydrochloric acid addition salt, hydrobromic acid which affords a hydrobromic acid addition salt, acetic acid which affords an acetic acid addition salt, ascorbic acid which affords an ascorbic acid addition salt, benzenesulfonic acid which affords a besylate addition salt, camphorsulfonic acid which affords a camphorsulfonic acid addition salt, citric acid which affords a citric acid addition salt, maleic acid which affords a maleic acid addition salt, malic acid which affords a malic acid addition salt, methanesulfonic acid which affords a methanesulfonic acid (mesylate) addition salt, naphthalenesulfonic acid which affords a naphthalenesulfonic acid addition salt, oxalic acid which affords an oxalic acid addition salt, phosphoric acid which affords a phosphoric acid addition salt, toluenesulfonic acid which affords a p-toluenesulfonic acid addition salt, succinic acid which affords a succinic acid addition salt, sulfuric acid which affords a sulfuric acid addition salt, tartaric acid which affords a tartaric acid addition salt and trifluoroacetic acid which affords a trifluoroacetic acid addition salt. Each of these acid addition salts can be either a mono-addition salt or a poly-addition salt, as these terms are defined herein.

As used herein, the term “prodrug” refers to a compound which is converted in the body to an active compound (e.g., the compound of the formula described hereinabove). A prodrug is typically designed to facilitate administration, e.g., by enhancing absorption. A prodrug may comprise, for example, the active compound modified with ester groups, for example, wherein any one or more of the hydroxyl groups of a compound is modified by an acyl group, optionally (C₁₋₄)-acyl (e.g., acetyl) group to form an ester group, and/or any one or more of the carboxylic acid groups of the compound is modified by an alkoxy or aryloxy group, optionally (C₁₋₄)-alkoxy (e.g., methyl, ethyl) group to form an ester group.

Further, each of the compounds described herein, including the salts thereof, can be in a form of a solvate or a hydrate thereof.

The term “solvate” refers to a complex of variable stoichiometry (e.g., di-, tri-, tetra-, penta-, hexa-, and so on), which is formed by a solute (the heterocyclic compounds described herein) and a solvent, whereby the solvent does not interfere with the biological activity of the solute.

The term “hydrate” refers to a solvate, as defined hereinabove, where the solvent is water.

The compounds described herein can be used as polymorphs and the present embodiments further encompass any isomorph of the compounds and any combination thereof.

The compounds and structures described herein encompass any stereoisomer, including enantiomers and diastereomers, of the compounds described herein, unless a particular stereoisomer is specifically indicated.

As used herein, the term “enantiomer” refers to a stereoisomer of a compound that is superposable with respect to its counterpart only by a complete inversion/reflection (mirror image) of each other. Enantiomers are said to have “handedness” since they refer to each other like the right and left hand. Enantiomers have identical chemical and physical properties except when present in an environment which by itself has handedness, such as all living systems. In the context of the present embodiments, a compound may exhibit one or more chiral centers, each of which exhibiting an (R) or an (S) configuration and any combination, and compounds according to some embodiments of the present invention, can have any their chiral centers exhibit an (R) or an (S) configuration.

The term “diastereomers”, as used herein, refers to stereoisomers that are not enantiomers to one another. Diastereomerism occurs when two or more stereoisomers of a compound have different configurations at one or more, but not all of the equivalent (related) stereocenters and are not mirror images of each other. When two diastereoisomers differ from each other at only one stereocenter they are epimers. Each stereo-center (chiral center) gives rise to two different configurations and thus to two different stereoisomers. In the context of the present invention, embodiments of the present invention encompass compounds with multiple chiral centers that occur in any combination of stereo-configuration, namely any diastereomer.

In any of the methods and uses described herein, a compound as described herein in any of the respective embodiments (capable of upregulating PERK activity), can be administered to an organism per se, or in a pharmaceutical composition that further comprises a pharmaceutically acceptable carrier.

As used herein a “pharmaceutical composition” refers to a preparation of one or more of the active ingredients described herein with other chemical components such as physiologically suitable carriers and excipients. The purpose of a pharmaceutical composition is to facilitate administration of a compound to an organism.

Herein the term “active ingredient” refers to the compound accountable for the biological effect (herein, a compound capable of upregulating PERK activity).

Hereinafter, the phrases “physiologically acceptable carrier” and “pharmaceutically acceptable carrier”, which may be interchangeably used, refer to a carrier or a diluent that does not cause significant irritation to an organism and does not abrogate the biological activity and properties of the administered compound. An adjuvant is included under these phrases.

Herein the term “excipient” refers to an inert substance added to a pharmaceutical composition to further facilitate administration of an active ingredient. Examples, without limitation, of excipients include calcium carbonate, calcium phosphate, various sugars and types of starch, cellulose derivatives, gelatin, vegetable oils and polyethylene glycols.

Techniques for formulation and administration of drugs may be found in “Remington's Pharmaceutical Sciences,” Mack Publishing Co., Easton, PA, latest edition, which is incorporated herein by reference.

Suitable routes of administration may, for example, include oral, rectal, topical, transmucosal, especially transnasal, intestinal or parenteral delivery, including intramuscular, subcutaneous and intramedullary injections as well as intrathecal, direct intraventricular, intracardiac, e.g., into the right or left ventricular cavity, into the common coronary artery, intravenous, intraperitoneal, intranasal, or intraocular injections.

Alternately, one may administer the pharmaceutical composition in a local rather than systemic manner, for example, via injection of the pharmaceutical composition directly into a tissue region of a patient.

The term “tissue” refers to part of an organism consisting of cells designed to perform a function or functions. Examples include, but are not limited to, brain tissue, retina, skin tissue, hepatic tissue, pancreatic tissue, bone, cartilage, connective tissue, blood tissue, muscle tissue, cardiac tissue brain tissue, vascular tissue, renal tissue, pulmonary tissue, gonadal tissue, hematopoietic tissue.

Pharmaceutical compositions of some embodiments of the invention may be manufactured by processes well known in the art, e.g., by means of conventional mixing, dissolving, granulating, dragee-making, levigating, emulsifying, encapsulating, entrapping or lyophilizing processes.

Pharmaceutical compositions for use in accordance with some embodiments of the invention thus may be formulated in conventional manner using one or more physiologically acceptable carriers comprising excipients and auxiliaries, which facilitate processing of the active ingredients into preparations which, can be used pharmaceutically. Proper formulation is dependent upon the route of administration chosen.

For topical administration, an appropriate carrier may be selected and optionally other ingredients that can be included in the composition, as is detailed herein. Hence, the compositions can be, for example, in a form of a cream, an ointment, a paste, a gel, a lotion, and/or a soap.

Ointments are semisolid preparations, typically based on vegetable oil (e.g., shea butter and/or cocoa butter), petrolatum or petroleum derivatives. As with other carriers or vehicles, an ointment base should be inert, stable, nonirritating and non-sensitizing.

Lotions are preparations that may to be applied to the skin without friction. Lotions are typically liquid or semiliquid preparations with a water or alcohol base, for example, an emulsion of the oil-in-water type. Lotions are typically preferred for treating large areas (e.g., as is frequently desirable for sunscreen compositions), due to the ease of applying a more fluid composition.

Creams are viscous liquids or semisolid emulsions, either oil-in-water or water-in-oil. Cream bases typically contain an oil phase, an emulsifier and an aqueous phase. The oil phase, also called the “lipophilic” phase, optionally comprises petrolatum and/or a fatty alcohol such as cetyl or stearyl alcohol. The aqueous phase optionally contains a humectant. The emulsifier in a cream formulation is optionally a nonionic, anionic, cationic or amphoteric surfactant.

Herein, the term “emulsion” refers to a composition comprising liquids in two or more distinct phases (e.g., a hydrophilic phase and a lipophilic phase). Non-liquid substances (e.g., dispersed solids and/or gas bubbles) may optionally also be present.

As used herein and in the art, a “water-in-oil emulsion” is an emulsion characterized by an aqueous phase which is dispersed within a lipophilic phase.

As used herein and in the art, an “oil-in-water emulsion” is an emulsion characterized by a lipophilic phase which is dispersed within an aqueous phase.

Pastes are semisolid dosage forms which, depending on the nature of the base, may be a fatty paste or a paste made from a single-phase aqueous gel. The base in a fatty paste is generally petrolatum, hydrophilic petrolatum, and the like. The pastes made from single-phase aqueous gels generally incorporate carboxymethylcellulose or the like as a base.

Gel formulations are semisolid, suspension-type systems. Single-phase gels optionally contain organic macromolecules distributed substantially uniformly throughout the carrier liquid, which is typically aqueous; but also, preferably, contains a non-aqueous solvent, and optionally an oil. Preferred organic macromolecules (e.g., gelling agents) include crosslinked acrylic acid polymers such as the family of carbomer polymers, e.g., carboxypolyalkylenes, that may be obtained commercially under the trademark Carbopol®. Other types of preferred polymers in this context are hydrophilic polymers such as polyethylene oxides, polyoxyethylene-polyoxypropylene copolymers and polyvinyl alcohol; cellulosic polymers such as hydroxypropyl cellulose, hydroxyethyl cellulose, hydroxypropyl methylcellulose, hydroxypropyl methylcellulose phthalate, and methyl cellulose; gums such as tragacanth and xanthan gum; sodium alginate; and gelatin. In order to prepare a uniform gel, dispersing agents such as alcohol or glycerin can be added, or the gelling agent can be dispersed by trituration, mechanical mixing or stirring, or combinations thereof.

A composition formulated for topical administration may optionally be present in a patch, a swab, a pledget, and/or a pad.

Dermal patches and the like may comprise some or all of the following components: a composition to be applied (e.g., as described herein); a liner for protecting the patch during storage, which is optionally removed prior to use; an adhesive for adhering different components together and/or adhering the patch to the skin; a backing which protects the patch from the outer environment; and/or a membrane which controls release of a drug to the skin.

Conventional approaches for drug delivery to the central nervous system (CNS) include: neurosurgical strategies (e.g., intracerebral injection or intracerebroventricular infusion); molecular manipulation of the agent (e.g., production of a chimeric fusion protein that comprises a transport peptide that has an affinity for an endothelial cell surface molecule in combination with an agent that is itself incapable of crossing the BBB) in an attempt to exploit one of the endogenous transport pathways of the BBB; pharmacological strategies designed to increase the lipid solubility of an agent (e.g., conjugation of water-soluble agents to lipid or cholesterol carriers); and the transitory disruption of the integrity of the BBB by hyperosmotic disruption (resulting from the infusion of a mannitol solution into the carotid artery or the use of a biologically active agent such as an angiotensin peptide). However, each of these strategies has limitations, such as the inherent risks associated with an invasive surgical procedure, a size limitation imposed by a limitation inherent in the endogenous transport systems, potentially undesirable biological side effects associated with the systemic administration of a chimeric molecule comprised of a carrier motif that could be active outside of the CNS, and the possible risk of brain damage within regions of the brain where the BBB is disrupted, which renders it a suboptimal delivery method.

For injection, the active ingredients of the pharmaceutical composition may be formulated in aqueous solutions, preferably in physiologically compatible buffers such as Hank's solution, Ringer's solution, or physiological salt buffer. For transmucosal administration, penetrants appropriate to the barrier to be permeated are used in the formulation. Such penetrants are generally known in the art.

For oral administration, the pharmaceutical composition can be formulated readily by combining the active compounds with pharmaceutically acceptable carriers well known in the art. Such carriers enable the pharmaceutical composition to be formulated as tablets, pills, dragees, capsules, liquids, gels, syrups, slurries, suspensions, and the like, for oral ingestion by a patient. Pharmacological preparations for oral use can be made using a solid excipient, optionally grinding the resulting mixture, and processing the mixture of granules, after adding suitable auxiliaries if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carboxymethylcellulose; and/or physiologically acceptable polymers such as polyvinylpyrrolidone (PVP). If desired, disintegrating agents may be added, such as cross-linked polyvinylpyrrolidone, agar, or alginic acid or a salt thereof such as sodium alginate.

Dragee cores are provided with suitable coatings. For this purpose, concentrated sugar solutions may be used which may optionally contain gum arabic, talc, polyvinyl pyrrolidone, carbopol gel, polyethylene glycol, titanium dioxide, lacquer solutions and suitable organic solvents or solvent mixtures. Dyestuffs or pigments may be added to the tablets or dragee coatings for identification or to characterize different combinations of active compound doses.

Pharmaceutical compositions which can be used orally, include push-fit capsules made of gelatin as well as soft, sealed capsules made of gelatin and a plasticizer, such as glycerol or sorbitol. The push-fit capsules may contain the active ingredients in admixture with filler such as lactose, binders such as starches, lubricants such as talc or magnesium stearate and, optionally, stabilizers. In soft capsules, the active ingredients may be dissolved or suspended in suitable liquids, such as fatty oils, liquid paraffin, or liquid polyethylene glycols. In addition, stabilizers may be added. All formulations for oral administration should be in dosages suitable for the chosen route of administration.

For buccal administration, the compositions may take the form of tablets or lozenges formulated in conventional manner.

For administration by nasal inhalation, the active ingredients for use according to some embodiments of the invention are conveniently delivered in the form of an aerosol spray presentation from a pressurized pack or a nebulizer with the use of a suitable propellant, e.g., dichlorodifluoromethane, trichlorofluoromethane, dichloro-tetrafluoroethane or carbon dioxide. In the case of a pressurized aerosol, the dosage unit may be determined by providing a valve to deliver a metered amount. Capsules and cartridges of, e.g., gelatin for use in a dispenser may be formulated containing a powder mix of the compound and a suitable powder base such as lactose or starch.

The pharmaceutical composition described herein may be formulated for parenteral administration, e.g., by bolus injection or continuous infusion. Formulations for injection may be presented in unit dosage form, e.g., in ampoules or in multidose containers with optionally, an added preservative. The compositions may be suspensions, solutions or emulsions in oily or aqueous vehicles, and may contain formulatory agents such as suspending, stabilizing and/or dispersing agents.

Pharmaceutical compositions for parenteral administration include aqueous solutions of the active preparation in water-soluble form. Additionally, suspensions of the active ingredients may be prepared as appropriate oily or water based injection suspensions. Suitable lipophilic solvents or vehicles include fatty oils such as sesame oil, or synthetic fatty acids esters such as ethyl oleate, triglycerides or liposomes. Aqueous injection suspensions may contain substances, which increase the viscosity of the suspension, such as sodium carboxymethyl cellulose, sorbitol or dextran. Optionally, the suspension may also contain suitable stabilizers or agents which increase the solubility of the active ingredients to allow for the preparation of highly concentrated solutions.

Alternatively, the active ingredient may be in powder form for constitution with a suitable vehicle, e.g., sterile, pyrogen-free water based solution, before use.

The pharmaceutical composition of some embodiments of the invention may also be formulated in rectal compositions such as suppositories or retention enemas, using, e.g., conventional suppository bases such as cocoa butter or other glycerides.

In some embodiments of any of the embodiments described herein, the use of the compound as described herein is effected in vivo, for example, by administering a therapeutically effective amount of the compound to a subject in need thereof.

Pharmaceutical compositions suitable for use in context of some embodiments of the invention include compositions wherein the active ingredients are contained in an amount effective to achieve the intended purpose. More specifically, a therapeutically effective amount means an amount of active ingredient (e.g., a compound according to any of the respective embodiments described herein) effective to prevent, alleviate or ameliorate symptoms of a disorder (e.g., a viral infection, or a disease or disorder associated with a viral infection) or prolong the survival of the subject being treated.

In some embodiments, the use of the compound as described herein is effected ex vivo (e.g., in vitro), for example, in research.

Determination of a therapeutically effective amount is well within the capability of those skilled in the art, especially in light of the detailed disclosure provided herein.

For any preparation used in the methods of the invention, the therapeutically effective amount or dose can be estimated initially from in vitro and cell culture assays. For example, a dose can be formulated in animal models to achieve a desired concentration or titer. Such information can be used to more accurately determine useful doses in humans.

Toxicity and therapeutic efficacy of the active ingredients described herein can be determined by standard pharmaceutical procedures in vitro, in cell cultures or experimental animals. The data obtained from these in vitro and cell culture assays and animal studies can be used in formulating a range of dosage for use in human. The dosage may vary depending upon the dosage form employed and the route of administration utilized. The exact formulation, route of administration and dosage can be chosen by the individual physician in view of the patient's condition. (See e.g., Fingl, et al., 1975, in “The Pharmacological Basis of Therapeutics”, Ch. 1 p. 1).

Dosage amount and interval may be adjusted individually to provide levels (e.g., blood levels) of the active ingredient sufficient to induce or suppress the biological effect (minimal effective concentration, MEC). The MEC will vary for each preparation, but can be estimated from in vitro data. Dosages necessary to achieve the MEC will depend on individual characteristics and route of administration. Detection assays can be used to determine plasma concentrations.

Depending on the severity and responsiveness of the condition to be treated, dosing can be of a single or a plurality of administrations, with course of treatment lasting from several days to several weeks or until cure is effected or diminution of the disease state is achieved.

The amount of a composition to be administered will, of course, be dependent on the subject being treated, the severity of the affliction, the manner of administration, the judgment of the prescribing physician, etc.

Compositions of some embodiments of the invention may, if desired, be presented in a pack or dispenser device, such as an FDA approved kit, which may contain one or more unit dosage forms containing the active ingredient. The pack may, for example, comprise metal or plastic foil, such as a blister pack. The pack or dispenser device may be accompanied by instructions for administration. The pack or dispenser may also be accommodated by a notice associated with the container in a form prescribed by a governmental agency regulating the manufacture, use or sale of pharmaceuticals, which notice is reflective of approval by the agency of the form of the compositions or human or veterinary administration. Such notice, for example, may be of labeling approved by the U.S. Food and Drug Administration for prescription drugs or of an approved product insert. Compositions comprising a preparation of the invention formulated in a compatible pharmaceutical carrier may also be prepared, placed in an appropriate container, and labeled for treatment of an indicated condition, as is further detailed above.

As used herein, the term “subject” includes mammals, preferably human beings at any age which suffer from the pathology. Preferably, this term encompasses individuals who are at risk to develop the pathology.

It is expected that during the life of a patent maturing from this application many relevant types of viral infections and viral strains will be developed and the scope of the term “infection” is intended to include all such new technologies a priori.

As used herein the term “about” refers to ±10% or ±5%.

The terms “comprises”, “comprising”, “includes”, “including”, “having” and their conjugates mean “including but not limited to”.

The term “consisting of” means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular form “a”, “an” and “the” include plural references unless the context clearly dictates otherwise. For example, the term “a compound” or “at least one compound” may include a plurality of compounds, including mixtures thereof.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination or as suitable in any other described embodiment of the invention. Certain features described in the context of various embodiments are not to be considered essential features of those embodiments, unless the embodiment is inoperative without those elements.

Various embodiments and aspects of the present invention as delineated hereinabove and as claimed in the claims section below find experimental support in the following examples.

### EXAMPLES

Reference is now made to the following examples, which together with the above descriptions illustrate some embodiments of the invention in a non-limiting fashion.

### Materials and Experimental Methods

### Materials:

Endo H was obtained from New England Biolabs.

N-glycanase was obtained from Roche.

Mouse monoclonal anti-VSV-G antibodies were obtained from Kerafast Inc. or Santa Cruz Biotechnology®, rabbit polyclonal anti-H2b carboxy-terminal antibodies were those used in previous studies (Tolchinsky, S. et al. J. Biol. Chem. (1996) 271, 14496-14503) and mouse monoclonal anti-ACE2 were obtained from Santa Cruz.

Microwave irradiation experiments: Microwave irradiation experiments were performed using CEM Discover@SP machine, using the following setup parameters:

| Method type | Dynamic |
|---|---|
| Pressure limit | 250 PSI |
| Vessel Type | 10 ml |
| Temperature | 100° C. |
| Power | 100 W |
| Hold time (h:m:s) | 00:08:00 |
| PreMix | No |
| Stirring | High |
| Cooling | On |

LC-MS: Waters AutoPurification System analytical module equipped with SQD2 MS detector at the following conditions: a. LC: Waters XBridge BEH300 C4 (3.5 μm, 4.6 mm×100 mm) using a 8-minute gradient from 95:5 Water: acetonitrile (both with 0.1% formic acid) to acetonitrile; b. MS: scan mode 100-1000.

¹H and ¹³C NMR: ¹H and ¹³C NMR spectra were measured on Bruker 400 (400 MHz ¹H, 100 MHz ¹³C). Chemical shifts values (6) are reported in ppm (calibration of spectra to the residual peak of TMS: δ=0.0 ppm (s) for ¹H NMR; δ=0.0 ppm for ¹³C NMR if not mentioned otherwise). All the proton spectra reported as following: 6 value (multiplicity, J coupling constant (in Hz), number of nuclei). Multiplicity contractions used: (s)—singlet, (d)—doublet, (dd)—doublet of doublet, (t)—triplet, (q)—quartet, (m)—multiplet, and (br)—broad signal.

### Chemical Syntheses:

Preparation of 4-((2-(4,6-diphenylpyrimidin-2-yl)-2-methylhydrazineylidene) methyl)benzene-1,2-diol) (MK-28):

Into a 10 mL process vial, equipped with a stirring bar, 2-(1-methylhydrazino)-4,6-diphenylpyrimidinederivate (0.3 mmol, 1.0 equivalent), and 3,4-dihydroxybenzaldehyde (0.45 mmol, 1.5 equivalents), in 2 ml isopropanol with a catalytic amount of acetic acid at room temperature. The vial was fitted with a snap-on cap, and the solution stirred for 10 seconds. Then, the vial was placed in a CEM Discover@SP microwave with setup parameters as described hereinabove. The reaction mixture was cooled in the refrigerator overnight, the resultant yellow precipitate was filtered, washed with cold isopropanol and dried by lyophilization, to provide MK-28 (98 mg, 82% yield) as a Z:E mixture of about 1:1, in at least 98% HPLC purity at diode array. HPLC: RT 6.01 minutes, using a 8-minute gradient from 95:5 Water: acetonitrile (both with 0.1% formic acid) to acetonitrile. MS: ES+397.51 [M+H]. ¹H NMR (400 MHz, DMSO) δ 1H NMR (400 MHz, DMSO-d6) δ 9.20 (bs, 2H), 8.47-8.35 (m, 4H), 8.06 (s, 1H), 7.90 (s, 1H), 7.59 (m, 6H), 7.38 (d, J=1.9 Hz, 1H), 7.07 (dd, J=8.2, 1.9 Hz, 1H), 6.81 (d, J=8.1 Hz, 1H), 3.82 (s, 3H). ¹³C NMR (101 MHz, DMSO) δ 165.07, 161.08, 147.09, 145.88, 139.22, 137.37, 131.23, 129.17, 128.04, 127.60, 120.04, 115.90, 113.18, 104.12, 31.61.

Preparation of 4-((2-(4,6-diphenylpyrimidin-2-yl)hydrazineylidene)methyl) benzene-1,2-diol (GLB-7):

GLB-7 was synthesized according to the following two-step procedure, as follows.

Preparation of 2-hydrazineyl-4,6-diphenylpyrimidine (GLB7-Im-1):

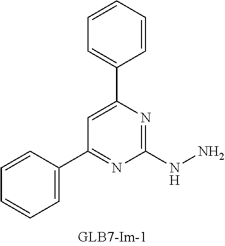 [SMILES: NNc1nc(-c2ccccc2)cc(-c2ccccc2)n1]

GLB7-Im-1 was prepared using the general Nucleophilic aromatic substitution described in Step 1 hereinabove, starting from 2-chloro-4,6-diphenylpyrimidine as compound A (0.6 mmol, 1.0 equivalent) and hydrazine monohydrate as compound B (20 mmol, 33 equivalents), to provide GLB7-Im-1 as compound C (119 mg, 75% yield) in at least 98% HPLC purity at diode array. HPLC: RT 5.38 minutes, using an 8-minute gradient from 95:5 Water: acetonitrile (both with 0.1% formic acid) to acetonitrile. MS: ESI+263.40 [M+H]. ¹H NMR (400 MHz, DMSO) δ 8.29 (m, 4H), 7.76 (s, 1H), 7.59-7.48 (m, 6H), 4.37 (s, 2H).

Preparation of 4-((2-(4,6-diphenylpyrimidin-2-yl)hydrazineylidene)methyl) benzene-1,2-diol (GLB7):

GLB7 was prepared using the general Schiff base reaction as described in Step 2 hereinabove, starting from GLB7-Im-1 as compound C (0.44 mmol, 1.0 equivalent), and 3,4-dihydroxybenzaldehyde (0.66 mmol, 1.5 equivalents) as compound D, in 2 ml isopropanol, to provide GLB7 (168 mg, 96% yield) as a Z:E mixture of about 1:1, in at least 98% HPLC purity at diode array. HPLC: RT 6.09 minutes, using an 8-minute gradient from 95:5 Water: acetonitrile (both with 0.1% formic acid) to acetonitrile. MS: ES+383.48 [M+H]. ¹H NMR (400 MHz, DMSO) δ 11.09 (s, 1H), 9.24 (s, 1H), 9.19 (s, 1H), 8.37-8.29 (m, 4H), 8.07 (s, 1H), 7.93 (s, 1H), 7.58 (m, 6H), 7.27 (d, J=1.8 Hz, 1H), 6.92 (dd, J=8.2, 1.8 Hz, 1H), 6.79 (dd, J=8.1, 1.5 Hz, 1H). ¹³C NMR (101 MHz, DMSO) δ 165.40, 160.94, 147.36, 146.03, 142.77, 131.15, 129.13, 127.57, 127.25, 119.88, 112.85, 104.23.

Viral strains: SARS-CoV-2 or VSVΔ51M, a mutated VSV which is unable to combat IFN released by normal cells but is still able to replicate in tumor cells, were used. The original VSV clone was obtained from Kerafast Inc., and the A51M mutation was inserted as described in Dellac et al. [Int J Cancer (2021) 148(9), 2321-2334].

Cell culture and infections: HEK293 and VERO cells were grown in DMEM supplemented with 10% bovine calf serum at 37° C. under 5% CO₂. Infection of VERO cells was as described, for example, in Dellac et al. [2021, supra].

Determination of viral titer: Viral titer was determined by plaque assay, carried out on Vero cells overlaid with tragacanth; as described, for example, in Dellac et al. [2021, supra].

Immunoblotting: Cell lysis and immunoblotting (western blotting) was performed according to procedures such as described in Ganz et al. [(2020) Scientific Reports, 10, 6875], using the indicated antibodies.

Plasmids and transfections: A plasmid expressing VSV-G, asialoglycoprotein receptor H2b was as described in (Tolchinsky, S. et al., J. Biol. Chem. (1996) 271, 14496-14503) and ACE2 was obtained from Sino Biological.

Transfections were carried out using the calcium phosphate method. HEK 293 cells were seeded 2 hours prior to transfection at 50-70% confluency. CaCl₂) solution containing desired DNA was mixed slowly with HEPES-buffered saline containing sodium phosphate. A DNA-calcium phosphate co-precipitate was added dropwise to the dishes after 20 minutes of incubation.

### Example 1

### Effect of Exemplary PERK Activator on SARS-CoV-2 Replication

In order to assess the effect of PERK activation on viral replication, the compound MK-28, an exemplary PERK activator [Ganz et al. (2020) Scientific Reports, 10, 6875] was investigated using a model of SARS-CoV-2 infected cells.

Vero E6 cells were treated with the exemplary compound MK-28 pre- and post-infection with SARS-CoV-2. The viral titer were measured at two intervals, of 16 and 24 hours post infection (hpi).

As shown in FIG. 1, treatment with the exemplary compound MK-28 reduced SARS-CoV-2 replication by about 10- to 100-fold.

These results indicate that PERK activation can result in antiviral activity.

### Example 2

### Effect of Exemplary PERK Activators on VSV Replication

In order to assess whether the exemplary PERK activator MK-28 exhibits antiviral activity against different viruses, and in order to examine the antiviral efficacy of MK-28 analogues such as GLB7, in vitro experiments were performed in cells infected with VSVΔ51M, a Δ51M mutant strain of the vesicular stomatitis Indiana virus (VSV).

HEK293 cells, untreated or treated with MK-28 or GLB7, were infected at different multiplicities of infection (0.1 or 1 MOI). At 24 hpi (hours post infection), supernatants of the infected cultures were collected and viral titers were measured.

As shown in FIG. 2, both MK-28 and GLB7 reduced the titer of infectious virions by several orders of magnitude at both MOIs, upon infection of HEK293 cells with VSVΔ51M. MK-28 exhibited greater inhibition of replication than did GLB7 at 0.1 MOI, whereas GLB7 exhibited greater inhibition than MK-28 at 1 MOI.

These results indicate that PERK activation by a variety of compounds can result in antiviral activity against a variety of viruses.

### Example 3

### Effect of MK-28 on ER Retention of Viral Glycoproteins

In order to investigate whether the antiviral activity exhibited by exemplary PERK activators in Examples 1 and 2 is associated with inhibition of the ER exit of viral glycoproteins (ER retention), the effect of MK-28 on VSV-G glycoproteins was analyzed.

The maturation degree of N-linked oligosaccharides of the VSV-G glycoprotein was analyzed by quantifying (by western blot) the ratio of Endo H-sensitive (ER-localized) and Endo H-resistant (Golgi/post-Golgi-localized) forms of the glycoprotein in VSVΔ51M-infected HEK293 lysate upon lysis 24 hours post-infection, following pre-incubation of the cells with MK-28.

As shown in FIGS. 3A and 3B, treatment of VSV-infected cells with MK-28 caused a considerable decrease in Endo H-resistance, to about half the resistance of untreated control cells (at an MOI of either 0.1 or 1), which is indicative of a decrease in VSV-G glycoprotein exiting the ER.

These results indicate that PERK activation promotes ER retention in cells infected by viruses, which inhibits viral maturation.

### Example 4

### Specificity of MK-28 Towards Viral Proteins

In view of the results presented in Example 3, the specificity of MK-28 in inhibiting maturation of viral proteins was examined. The degree by which MK-28 affects oligosaccharide maturation of cellular proteins was studied in HEK293 cells transfected with plasmid expressing either the viral VSV-G protein, or the cellular H2b or ACE2 proteins. Lysates were treated with Endo H (which cleaves only high mannose glycans) or with N-glycanase (which cleaves any N-glycan).

As shown in FIG. 4A, western blot analyses indicated that VSV-G is resistant to Endo H in untreated VSV-G transfected HEK293 cells, whereas Endo H-resistance decreased considerably in cells treated with MK-28 (FIG. 4A, right), indicating ER retention and inhibited maturation by MK-28.

However, as shown in FIGS. 4B and 4C, the cellular proteins H2b (FIG. 4B) and ACE2 (FIG. 4C) exhibited no effect following treatment with MK-28, which indicates that neither the maturation of the H2b asialoglycoprotein receptor subunit, nor that of the SARS-CoV-2 receptor ACE2, were impeded by MK-28 treatment.

These results indicate that inhibition of glycoprotein maturation by PERK activators is specific to viral proteins.

These results further suggest that the anti-SARS-CoV-2 effect of PERK activators (indicated in Example 1) stems from effects on virus replication, and not on trafficking defects of the ACE2 receptor.

Overall, these results indicate that activation of a UPR pathway hampers viruses. This approach acts independently from the immune response of the infected individual and from the specific viral variant involved; thereby allowing it to be applied in the treatment of a broad range of viral infections.

### Example 5

### Effect of MK-28 on PERK Activity Under Viral Infection In-Vitro

The ability of MK-28 to boost the activation of PERK under viral infection was assessed in-vitro.

For this purpose, HEK293 cells were infected with VSVΔ51M at 0.1 MOI. Concomitant with the infection, or starting 3 hours after the start of the infection, the cells were treated with MK-28. Cells were lysed 24 hours after start of the infection and the levels of phosphorylated eIF2α relative to total eIF2α (substrate of PERK) were measured.

As can be seen in FIGS. 5A, 5B and 5D, there was a strong increase in eIF2α phosphorylation (eIF2α-P) following treatment with either GLB7 or MK-28, which indicate PERK activation by these compounds.

The levels of VSV-G glycoprotein were also measured as an indicator of the levels of infection, and the results are presented in FIGS. 5A, 5C and 5E. As the decrease in VSV-G glycoprotein indicates, there was a decrease in infectivity.

When the treatment began at the start of the infection, 20 μM MK-28 caused about 5-fold increase in eIF2α phosphorylation and a about 20-fold decrease in infectivity (FIGS. 5B-C). Even when the treatment began 3 hours after the start of the infection, 20 μM MK-28 caused about 4-fold increase in eIF2α phosphorylation and about 4-fold decrease in infectivity (FIGS. 5D-E).

HEK293 cells were untreated or treated with 10 or 20 μM MK-28, the cells were lysed 24 hours post-treatment and the levels of phosphorylated eIF2α relative to total eIF2α were measured. The results, presented in FIG. 6, show that 10 μM MK-28 caused about 1.4-fold increase in eIF2α phosphorylation and 20 μM MK-28 caused about 1.1-fold increase.

When comparing the results from HEK293 cells (FIG. 6) and HEK293 cells infected with VSVΔ51M (FIGS. 5B and 5D), it can be concluded that MK-28 treatment leads to higher levels of phosphorylated eIF2α when the treated cells are under viral infection. This is possibly due to the activation of cytosolic PKR by the viral response, which is only partially neutralized by the virus, in addition to PERK activation by MK-28. This leads to additional phosphorylation of eIF2α, as its dephosphorylation is likely overwhelmed. It can therefore be deduced that the PERK activation by compounds as described herein is effected in infected cells at a higher extent compared to non-infected cells.

A similar experiment was performed to assess PERK activation and inhibition of viral infectivity by GLB7. HEK293 cells were treated with 10 or 20 μM GLB7 concomitantly with the infection, and the results are presented in FIGS. 7A-C.

As can be seen, there was a strong increase in eIF2α phosphorylation (FIG. 7A) indicating PERK activation and a major decrease in infectivity (FIG. 7C). In this case the cells were treated together with the start of the infection, 20 μM GLB7 caused about 1.5-fold increase in eIF2α phosphorylation and about 3-fold decrease in infectivity.

To conclude, the exemplary compounds activate PERK and decrease infectivity in viral-infected cells.

### Example 6

### Solubility and Lipophilicity

The solubility and lipophilicity of GLB7 and MK-28, two exemplary compounds according to some embodiments of the present invention, were measured.

The results are presented in Table 1. As can be seen, the exemplary compound GLB7 showed improved solubility compared to MK-28 (more than 10-fold higher). A decreased lipophilicity was observed for GLB7, which is typically a favorable characteristic when pursuing the design of novel drugs.

TABLE 1

| Compound | Solubility (μM) | Lipophilicity (logD_{7.4}) |
|---|---|---|
| MK-28 | 1.4 | 4.5 |
| GLB7 | 16 | 3.6 |

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

It is the intent of the applicant(s) that all publications, patents and patent applications referred to in this specification are to be incorporated in their entirety by reference into the specification, as if each individual publication, patent or patent application was specifically and individually noted when referenced that it is to be incorporated herein by reference. In addition, citation or identification of any reference in this application shall not be construed as an admission that such reference is available as prior art to the present invention. To the extent that section headings are used, they should not be construed as necessarily limiting. In addition, any priority document(s) of this application is/are hereby incorporated herein by reference in its/their entirety.

1-27. (canceled)

28. A method of treating a viral infection in a subject in need thereof, the method comprising administering to the subject a therapeutically affective amount of a compound capable of activating an unfolded protein response in a cell and/or of upregulating PERK activity, thereby treating the viral infection.

29. The method of claim 28, wherein the compound is a PERK activator.

30. The method of claim 28, wherein the compound is represented by Formula I: 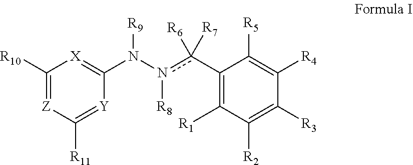
or a pharmaceutical acceptable salt thereof,
wherein:
the dashed line denotes a saturated or unsaturated bond;
X is N or CR₁₂;
Y is N or CR₁₃;
Z is N or CR₁₄;
R₁-R₇ and R₁₀-R₁₄ are each independently selected from the group consisting of hydrogen, alkyl, alkenyl, alkynyl, cycloalkyl, aryl, heteroaryl, heteroalicyclic, halo, hydroxy, alkoxy, aryloxy, thiohydroxy, thioalkoxy, thioaryloxy, sulfinyl, sulfonyl, sulfonate, sulfate, cyano, nitro, azide, phosphonyl, phosphinyl, carbonyl, thiocarbonyl, a urea group, a thiourea group, O-carbamyl, N-carbamyl, O-thiocarbamyl, N-thiocarbamyl, S-thiocarbamyl, C-amido, N-amido, C-carboxy, O-carboxy, sulfonamido, guanyl, guanidinyl, hydrazine, hydrazide, thiohydrazide, and amino; and
R₈ and R₉ are each independently hydrogen or alkyl,
wherein when the dashed line denotes an unsaturated bond, R₇ and R₈ are absent.

31. The method of claim 30, wherein R₁ is hydrogen or hydroxy.

32. The method of claim 30, wherein R₂-R₄ are each independently selected from hydrogen, hydroxy, alkoxy, and alkyl.

33. The method of claim 30, wherein at least one of R₂-R₄ is hydroxy.

34. The method of claim 30, wherein at least one of R₂-R₄ is alkoxy.

35. The method of claim 30, wherein at least one of R₂-R₄ is trifluoromethyl.

36. The method of claim 30, wherein the dashed line denotes a saturated bond and R₇ and R₈ are each hydrogen, or the dashed line denotes an unsaturated bond and R₇ and R₈ are each absent.

37. The method of claim 30, wherein the dashed line denotes an unsaturated bond.

38. The method of claim 30, wherein R₁₀ and R₁₁ are each independently selected from hydrogen, alkyl, cycloalkyl, aryl, heteroaryl, and heteroalicyclic.

39. The method of claim 30, wherein R₁₀ and R₁₁ are each independently a substituted or non-substituted phenyl.

40. The method of claim 30, wherein R₉ is hydrogen or methyl.

41. The method of claim 30, wherein at least one of X, Y and Z is nitrogen.

42. The method of claim 30, wherein X and Y are each nitrogen.

43. The method of claim 42, wherein Z is CH.

44. The method of claim 28, wherein the viral infection is associated with a virus selected from the group consisting of double strand DNA viruses, single strand DNA viruses, double strand RNA viruses, (+)-single strand RNA viruses, (−)-single strand RNA viruses, RNA retroviruses; DNA retroviruses, satellite viruses, and viroids.

45. The method of claim 44, wherein said virus is selected from the group consisting of coronaviruses, rhabdoviruses and reoviruses.

46. The method of claim 45, wherein said virus is a severe acute respiratory syndrome coronavirus (SARS-CoV) or a Vesicular stomatitis Indiana virus (VSV).

47. The method of claim 28, wherein the compound forms a part of a pharmaceutical composition which further comprises a pharmaceutically acceptable carrier.
